(19) United States
(12) Patent Application Publication
(10) Pub. No.: US 2010/0015091 A1
(43) Pub. Date: Jan. 21, 2010

(54) MODIFIED STAT1 TRANSGENE THAT CONFERS INTERFERON HYPERRESPONSIVENESS, METHODS AND USES THEREFOR

(75) Inventors: Michael J. Holtzman, St. Louis, MO (US); Yong Zhang, Chesterfield, MO (US)

Correspondence Address: WASHINGTON UNIVERSITY-SNR;C/O SONNENSCHEIN NATH & ROSENTHAL L.L.P, P.O. BOX 061080, WACKER DRIVE STATION , WILLIS TOWER, CHICAGO, IL, 60606, US

(73) Assignee: WASHINGTON UNIVERSITY IN ST. LOUIS, St. Louis, MO (US)

(21) Appl. No.: 12/504,612

(22) Filed: Jul. 16, 2009

Related U.S. Application Data
(60) Provisional application No. 61/135,104, filed on Jul. 16, 2008.

(51) Int. Cl. A61K 38/21; A61K 31/7088

(52) U.S. Cl. 424/85.4; 514/44 R

(57) ABSTRACT

Methods of enhancing cellular responses to interferons are disclosed. These methods comprise administering to a subject a vector comprising a Stat1-CC transgene, such as an AAV5 vector comprising a reporter operably linked to a nucleic acid sequence encoding a Stat1-CC polypeptide. The methods can be used in the treatment of diseases that involve interferon responses, such as multiple sclerosis, amyotrophic lateral sclerosis, and lupus; viral infections such as infection by hepatitis C virus, influenza A virus, cowpox virus, Sendai virus or Encephalomyocarditis virus; respiratory disorders; and cancers.

### CROSS-REFERENCE TO RELATED APPLICATIONS

This application claims priority from U.S. Provisional Application Ser. No. 61/135,104, filed on Jul. 16, 2008, which is incorporated herein by reference in its entirety.

### STATEMENT REGARDING FEDERALLY SPONSORED RESEARCH OR DEVELOPMENT

The disclosed subject matter was developed in part with Government support under grants P50HL056419-10 and U19AI070489-01 from the National Institutes of Health. The Government has certain rights in the invention.

### INCORPORATION-BY-REFERENCE OF SEQUENCE LISTING IN COMPUTER READABLE FORM

The Sequence Listing, which is a part of the present disclosure, includes a computer readable form comprising nucleotide and/or amino acid sequences of the present invention submitted via EFS-Web. The subject matter of the Sequence Listing is incorporated herein by reference in its entirety.

### INTRODUCTION

Viruses are among the most frequent causes of acute and chronic illness, and newly discovered viruses continue to cause emergent disease (van den Hoogen, B. G., et al. 2001. Nat. Med. 7:719-724; Kuiken, T., et al. 2003. Lancet 362:263-270). Despite the scope of this problem, current antiviral treatments aimed at crippling viral mechanisms for replication are quite limited in effectiveness. One alternative strategy to agents that target the viral machinery is to bolster the interferon (IFN) system (Sen, G. C. 2001. Annu. Rev. Microbiol. 55:255-281). However, targeting IFN efficacy is made difficult by the complexity in both its signaling pathway and its functional activities. At least 30 distinct IFN-induced genes may directly or indirectly control viral replication by regulating innate and adaptive immunity (Decker, T., et al. 2002. J. Clin. Invest. 109:1271-1277; Takaoka, A., et al. 2003. Nature 424:516-523; Tyner, J. W., et al. 2004. J. Allergy Clin. Immunol. 113:S49). In addition, the protective actions of IFNs are believed to rely on signaling through two IFN receptors (IFNAR for type I and IFNGR for type II IFNs, respectively) and the Janus kinase/signal transducer and activator of transcription (JAK-STAT) pathway (Schindler, C. 2002. J. Clin. Invest. 109:1133-1137). The latter pathway includes receptor-associated JAKs (Jak1, Jak2, Tyk2) and STATs (Stat1 and Stat2) as well as downstream transcription factors, enhancers, and coactivators. Despite the complexity in IFN signaling, the Stat1 transcription factor is believed to be common to both type I and type II IFN signaling pathways.

Overexpression or direct administration of IFN to influence viral infection in animal models has been attempted (Horwitz, M. S., et al. 2000. Nat. Med. 6:693-697; Haagmans, B. L., et al. 2004. Nat. Med. 10:290-293). Delivery of IFN has been used for viral infections in humans as well (Manns, M. P., et al. 2001. Lancet 358:958-965). However, viruses exhibit variable susceptibility to type I versus type II IFN, and the toxicity of IFN therapy has limited its effectiveness for treatment of viral infections in humans (Borden, E. C., et al. 2007. Nat. Rev. Drug Discov. 6:975-990). For example, cardiac expression of a dominant-negative SOCS1 (an endogenous inhibitor of Stat1 phosphorylation) protected against focal Coxsackie B virus-induced injury to the heart, but did not determine the effect on host viral clearance or outcome (Yasukawa, H., et al. 2003. J. Clin. Invest. 111:469-478). We found that increasing Stat1 levels (either by IFN-α priming or plasmid-mediated Stat1 expression) had little effect on subsequent IFN stimulation (Sampath, D., et al. 1998. FASEB J. 12:A1390). Furthermore, overactivity of the interferon system might drive cytopathic effects that may be detrimental in some settings, and constitutive activation of Stat1 may be associated with inflammatory disease (Sampath, D., et al. 1999. J. Clin. Invest. 103:1353-1361).

### SUMMARY

In view of a need for alternative therapeutic strategies, the present inventor has developed methods and compositions for introducing a double cysteine-substituted Stat1 (designated Stat1-CC) into cells in vivo.

Thus, in various aspects, the present inventor provides methods of treating a viral infection, methods of inducing expression of at least one IFN-responsive gene in at least one cell in vivo, methods of treating an interferon-responsive disease, and methods of protecting a subject from viral infection. In these aspects, the methods comprise administering to a subject, a vector comprising a Stat1-CC transgene.

In various configurations, a vector can be any type of vector known to skilled artisans, such as, without limitation, a plasmid or a virus. In some configurations, a viral vector can be an adeno-associated virus (AAV) such as, but not limited to, an AAV5. In some configurations, a vector can further comprise a promoter operably linked to the Stat1-CC transgene. The promoter can be any promoter known to skilled artisans, such as, but not limited to, a CMV-β-actin promoter.

In various configurations, following administration of the vector, one or more cells comprised by the subject can express the Stat1-CC transgene.

When a method of the present teachings is applied to treating a viral infection, the viral infection can be of a virus which induces a cellular interferon response, such as, without limitation, an encephalomyocarditis virus (EMCV), a hepatitis virus B virus, a hepatitis C virus, a vesicular stomatitis virus (VSV), a pneumovirus, a coronavirus, a coxsackievirus, or an enterovirus. In some configurations, a subject administered a vector of the present teachings can exhibit an increased rate of viral clearance compared to a control which is not administered the vector.

In addition, in some aspects, at least one cell comprised by a subject administered a vector of the present teachings can express the Stat1-CC transgene, and exhibit increased activation of an interferon, such as IFN-β. Furthermore, a cell that expresses the Stat1-CC transgene can exhibit enhanced efficiency of activation of one or more interferon-responsive genes, compared to a cell of a control that does not express the Stat1-CC transgene.

In some additional aspects, a subject which is administered a vector of the present teachings can exhibit a decreased rate of viral spread among neighboring cells and/or a decrease rate of viral replication, compared to a control subject which is not administered the vector.

In various configurations, one or more cells which express the Stat1-CC transgene in a subject administered a vector of the present teachings can be a cell of any organ or tissue of the subject, such as, without limitation, pancreas, brain or heart.

In various configurations, one or more cells which express the Stat1-CC transgene in a subject administered a vector of the present teachings can comprise a Stat1-CC transgene product which can exhibit prolonged Tyr-701 phosphorylation in response to IFN-γ treatment and/or can exhibit prolonged nuclear localization in response to IFN-γ treatment, compared to cells which express only wild-type Stat1.

In various configurations, one or more cells which express the Stat1-CC transgene in a subject administered a vector of the present teachings can express the Stat1-CC transgene and exhibit increased IFN efficacy upon administration of IFN, compared to cells which express only wild-type Stat1.

In some configurations of the present methods for inducing increased expression of at least one IFN-responsive gene in vivo in at least one cell comprised by a subject, the at least one IFN-responsive gene can be at least one type I IFN-responsive gene, such as, without limitation, an OAS, an Mx-1, and/or an MHC-I.

In some aspects of the present teachings, a method can further comprise administering an IFN to a subject, such as, without limitation, an IFN-β. Furthermore, when a subject is administered an IFN in addition to a vector of the present teachings, at least one cell of the subject can exhibit enhanced expression of at least one type I IFN-responsive gene, at least one type II IFN-responsive gene or a combination thereof. In these aspects, some non-limiting type I IFN-responsive genes include an OAS, an Mx-1, and an MHC-I, and non-limiting type II IFN-responsive genes can include an ICAM-1.

An interferon-responsive disease which can be treated by the methods disclosed herein can include any interferon-responsive disease or disorder known to skilled artisans, such as, without limitation, multiple sclerosis, amyotrophic lateral sclerosis, lupus, hepatitis C infection, a respiratory disorder or a cancer. Without limitation, a respiratory disorder, which can be treated by the disclosed methods, can include an interstitial lung disease, a malignant mesothelioma, a malignant pleural effusion, or a respiratory infection. Furthermore, examples of cancers, which can be treated by the disclosed methods, can include, without limitation, a hairy cell leukemia, a malignant melanoma, a Kaposi's sarcoma, a bladder cancer, a chronic myelocytic leukemia, a kidney cancer, a non-Hodgkin's lymphoma, a lung cancer, an ovarian cancer, and a skin cancer. In various configurations, these methods can include administering to a subject in need of treatment an effective dose of a vector disclosed herein, and, in some configurations, can further comprise administering an effective dose of an interferon to the subject. In some configurations, an effective dose of an interferon can be less than an effective dose of the interferon without administering the vector. In various configurations, administering an interferon can be simultaneous with administration of a vector, prior to administration of a vector, or following administration of a vector.

In some alternative configurations, a method of the present teachings can also comprise administering a vector and administering an inducer of expression of an interferon. In some configurations, an effective dose of an interferon inducer can be less than an effective dose of the interferon inducer without administering the vector. In various configurations, administering an interferon inducer can be simultaneous with administration of a vector, prior to administration of a vector, or following administration of a vector.

In various configurations, a subject can be a mammal, such as, without limitation a human, a companion animal such as a dog or cat, a farm animal such as a cow, a goat, a pig or a sheep, or a laboratory animal such as a mouse, a rat, a rabbit, or a guinea pig.

In aspects of the present teachings, which set forth methods of protecting a subject from a viral infection, the subject can comprise one or more cells which express the Stat1-CC transgene following administration of the vector.

### BRIEF DESCRIPTION OF THE DRAWINGS

FIG. 1 illustrates improved control of viral replication in Stat1-CC-expressing 2fTGH cells.

FIG. 2 illustrates Stat1-CC transgene expression and activation.

FIG. 3 illustrates enhanced IFN efficacy for gene expression in Stat1-CC transgenic mice.

FIG. 4 illustrates protection against viral infection in Stat1-CC transgenic mice.

FIG. 5 illustrates protection against EMCV infection and consequent encephalitis and myocarditis in Stat1-CC transgenic mice.

FIG. 6 illustrates effect of bone marrow transfer on susceptibility to EMCV infection.

FIG. 7 illustrates enhanced IFN-dependent gene expression in Stat1-CC-expressing human U3A cells.

FIG. 8 illustrates enhanced control of viral replication in Stat1-CC-expressing human U3A cells.

FIG. 9 illustrates protection against influenza A virus (IAV) infection in Stat1-CC transgenic mice.

FIG. 10 illustrates protection against influenza virus infection in rCCSP-Stat1-CC transgenic mice.

FIG. 11 illustrates protection against IAV infection in AAV5-Stat1-CC treated mice.

FIG. 12 illustrates decreased influenza virus levels in Stat1-CC-expressing cells.

FIG. 13 illustrates protection against Sendai virus (SeV) in Stat1-CC transgenic mice.

FIG. 14 illustrates protection against chronic lung disease in Stat1-CC Transgenic mice.

FIG. 15 illustrates increased interferon-induced apoptosis in Stat1-CC expressing U3A cells.

FIG. 16 illustrates decreased tumor formation by Stat1-CC-expressing U3A cells.

### DETAILED DESCRIPTION

The present teachings disclose methods of enhancing expression of interferon-responsive genes in vivo. The methods involve introducing into cells of a subject a vector comprising a promoter operably linked to a nucleic acid sequence encoding a Stat1-CC. The vectors can thus be used to treat interferon-responsive diseases, including viral infections.

### Suppression of Viral Replication in Stat1-CC-Transduced Cells

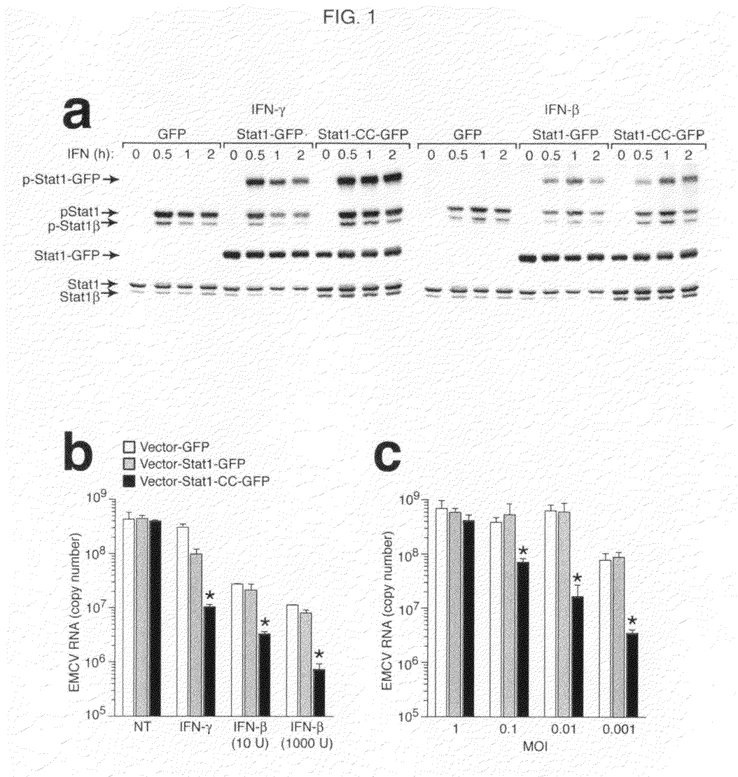

We previously found that enhanced IFN efficacy translated into improved antiviral action in Stat1-CC-versus Stat1-expressing or Stat1-null U3A cells that were pre-treated with IFN and then infected with EMCV (Zhang, Y., et al. 2005. J. Biol. Chem. 280:34306-34315). Here we extend those findings in two ways. First, we show that expression of Stat1-CC confers better viral clearance in U3A parental 2fTGH cells that contain endogenous Stat1 (FIG. 1a,b). These findings indicate that IFN-β activation of Stat1-CC protects cells that are not yet infected with virus. Therefore, the protective effects of Stat1 can be more evident at lower MOI that allows for viral spread to neighboring cells. Indeed, we found a significant decrease in viral replication rates in Stat1-CC-expressing cells compared to Stat1-expressing or native 2fTGH cells (FIG. 1c). Furthermore, we found no benefit for viral clearance by expressing wild-type Stat1 in 2fTGH cells. These results indicate that endogenous levels of Stat1 do not limit the antiviral response, whereas Stat1-CC, by providing more efficient activation of IFN-responsive genes, can improve the antiviral response.

### IFN Hyperresponsiveness in Stat1-CC Transgenic Mice

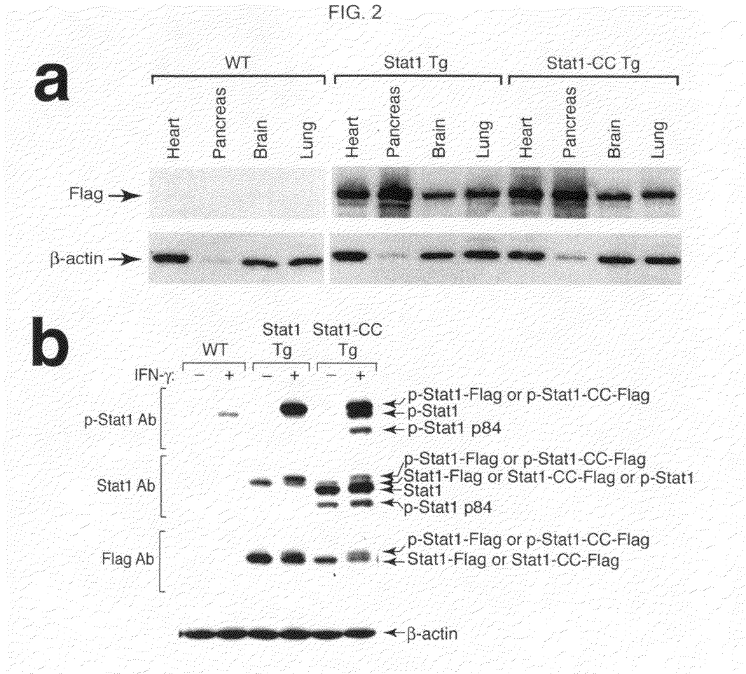

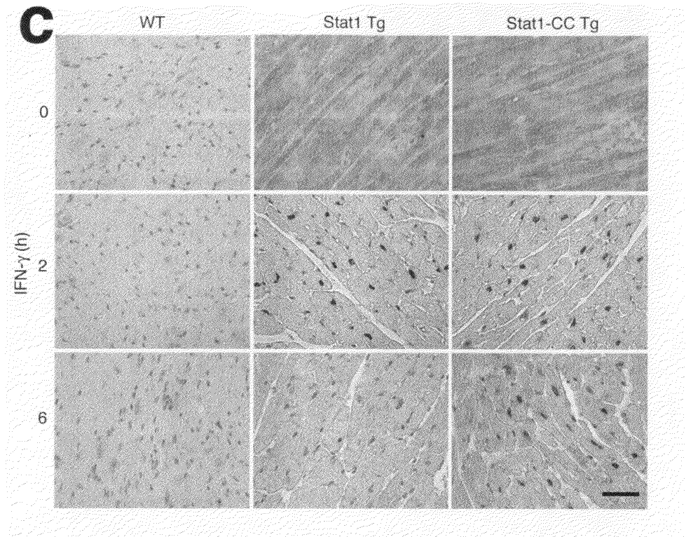

The results from Stat1-CC-transduced cells suggested that Stat1-CC expression in host cells can also enhance antiviral defenses in vivo. Accordingly, we generated transgenic mice with the CMV-β-actin promoter driving wild-type Stat1-3×Flag or Stat1-CC-3×Flag. Three of five founders carrying the wild-type Stat1 expression cassette and two of four founders carrying the Stat1-CC cassette expressed the predicted Stat1 or Stat1-CC transgene based on Western blotting. We found high-level transgene expression in various tissues, such as heart, pancreas, and skeletal muscle tissues, and intermediate-level expression in brain, lung, thymus, and spleen (see, e.g., FIG. 2a). We also found that Stat1 and Stat1-CC transgenic mice followed Mendelian rules for reproduction and exhibited no detectable development defects.

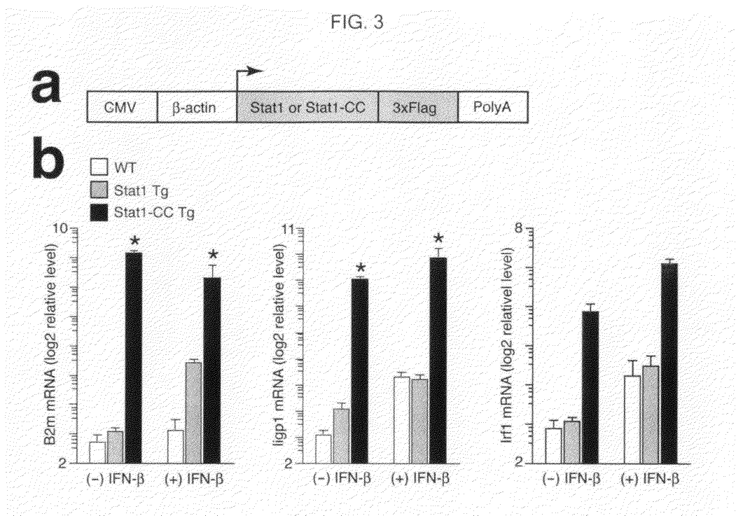

We next assessed the function of Stat1- and Stat1-CC transgene products in vivo. Similar to behavior in Stat1-CC-expressing cell lines, we found that the Stat1-CC transgene product also exhibited prolonged Tyr-701 phosphorylation and nuclear localization in response to IFN-γ treatment compared to wild-type Stat1 (FIG. 2b,c). Moreover, Stat1-CC transgenic mice exhibited increased gene expression in response to injected IFN-γ and IFN-β (FIG. 3b and data not shown), indicating that Stat1-CC conferred increases in IFN efficacy in vivo similar to those found in vitro. However, we also detected marked increases in baseline gene expression without IFN treatment (FIG. 3b and data not shown). Because Stat1-CC requires ligand-dependent phosphorylation for function (Zhang, Y., et al. 2005. J. Biol. Chem. 280:34306-34315), these findings indicate that low-level production of type I IFN is able to drive Stat1-CC activation and consequent increases in gene expression in vivo even under baseline conditions.

As further developed below, Stat1-CC transgenic mice exhibited an expression profile that could be broadly grouped into IFN-responsive genes that contribute to antiviral defense directly through the innate immune response (especially by inhibition of viral replication) and indirectly through the adaptive immune response (especially by antigen processing and presentation).

Protection from Viral Infection in Stat1-CC Transgenic Mice.

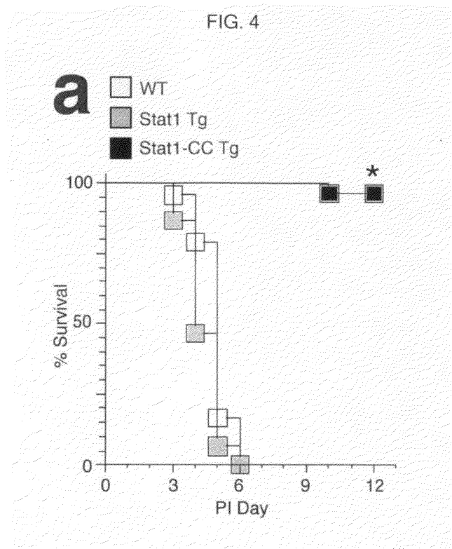

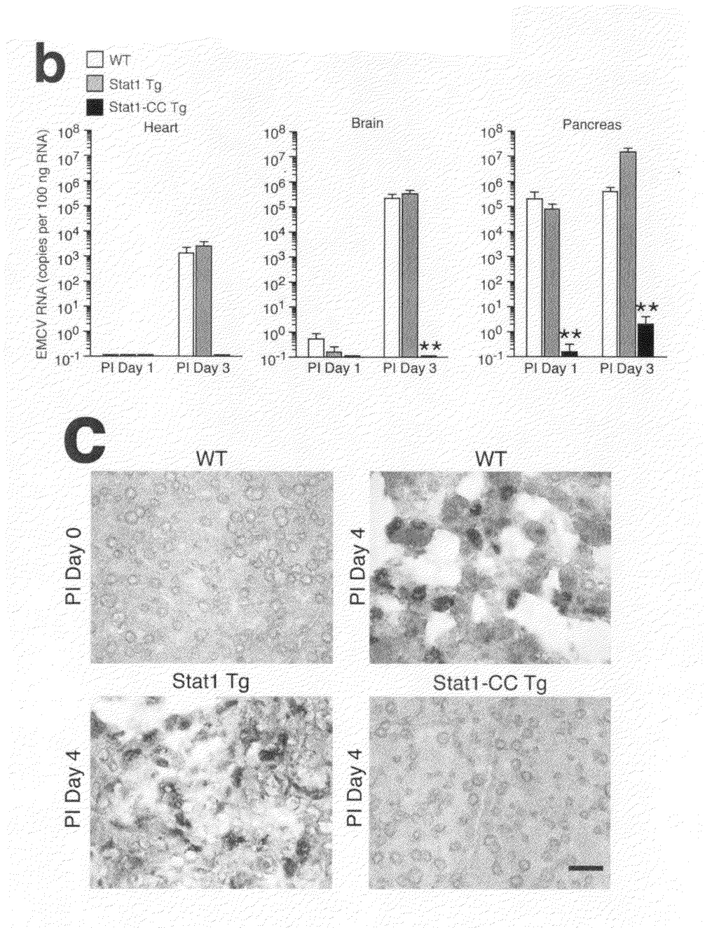

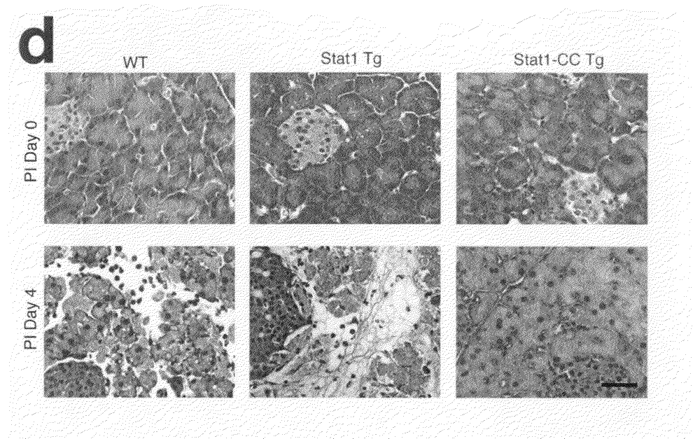

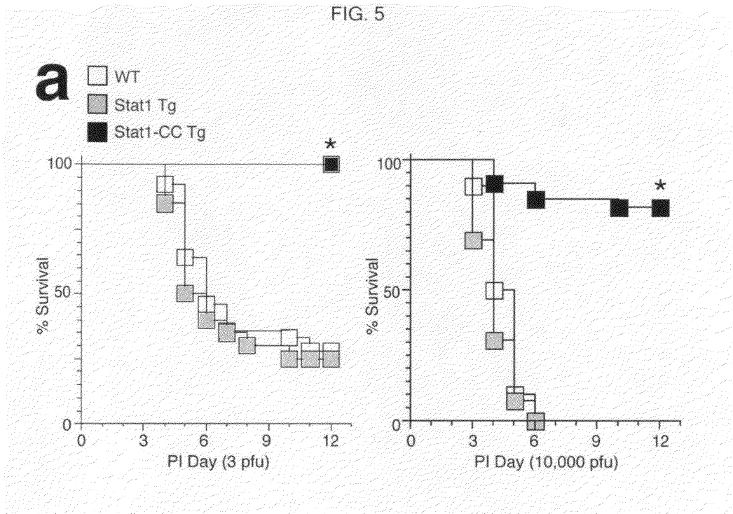

We found that CMV-b-actin-Stat1-CC transgenic mice are also markedly protected from viral infection. Inoculation with EMCV at 100 pfu caused a uniformly lethal infection in wild-type C57BL/6J mice as well as Stat1 transgenic mice (FIG. 4a). By contrast, Stat1-CC transgenic mice survived at a rate of 97% at this viral inoculum and at a rate of 82% even at 100-fold higher inoculum (FIGS. 4a and 5a). At lower viral inoculum of 3 pfu, wild-type and Stat1 transgenic mice survived at a rate of 25-28% whereas Stat1-CC mice survived at a rate of 100% (FIG. 5a). The improved survival rate was associated with a marked decrease in viral titers in heart, brain, and pancreas in Stat1-CC transgenic mice (FIG. 4b). Similarly, we found decreased levels of EMCV by immunostaining in pancreas in Stat1-CC transgenic mice compared to wild-type or Stat1 transgenic mice (FIG. 4c).

Necropsy indicated that EMCV tissue damage occurred in concert with the sites of viral replication. Thus, the major site of injury appeared to be the pancreas (where we detected the highest viral titers), followed by brain and heart. Tissue sections showed severe edema, damage, and inflammatory cell infiltration in wild-type and Stat1 transgenic mice after EMCV infection (FIG. 4d). By contrast, pancreas tissue exhibited only little of these abnormalities in Stat1-CC transgenic mice infected with EMCV. The major site of viral damage to the pancreas was localized to exocrine tissue, with relative sparing of islet tissue.

Similar to the case for pancreas, we found a marked decrease in encephalitis in Stat1-CC transgenic mice after EMCV infection. Thus, we found neuronal shrinkage and necrosis in the brains of wild-type and Stat1 transgenic mice, whereas these pathological alterations were not observed in Stat1-CC transgenic mice (FIG. 5b).

We also detected the development of a dilated cardiomyopathy based on gross pathology at necropsy as well as echocardiography in a subgroup of wild-type and Stat1 transgenic mice (data not shown). In addition, we found mild inflammation and edema in myocardial tissue in wild-type or transgenic mice after EMCV infection (FIG. 5c). The observed changes in myocardial function were therefore most likely due to toxicity of the infection as well as a low level of viral replication at this site. These abnormalities in myocardial function and histology were not detected in Stat1-CC transgenic mice infected with EMCV.

Taken together, the findings indicate that expression of the Stat1-CC transgene allows the host to achieve lower levels of virus and virus-induced tissue damage in various organs, including heart, brain, and pancreas.

### Stat1-CC Controls Viral Replication at the Tissue Host Cell Level

Our studies of transduced cells indicated that Stat1-CC provides a beneficial effect by enhancing innate immune control of viral replication in neighboring host cells. However, our gene expression analysis indicated that Stat1-CC might also act through IFN-responsive genes that mediate the adaptive immune response in vivo. Thus, either host cell suppression of viral replication or immune cell enhancement of antigen presentation could be responsible for the improved outcome in Stat1-CC transgenic mice. Accordingly, we next aimed to test whether expression of Stat1-CC in host tissue cells versus immune cells can be protective after viral infection in vivo.

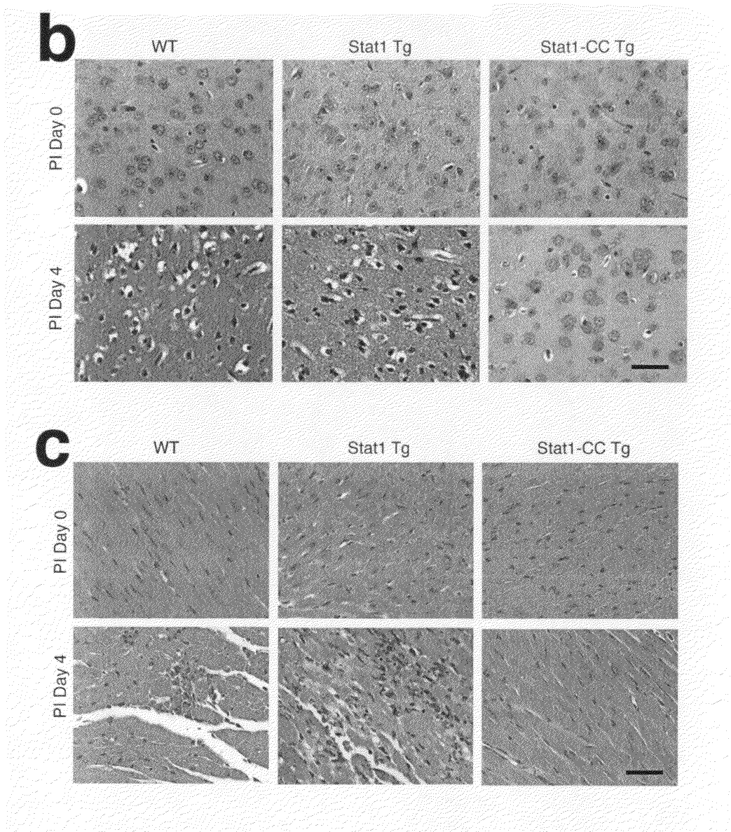

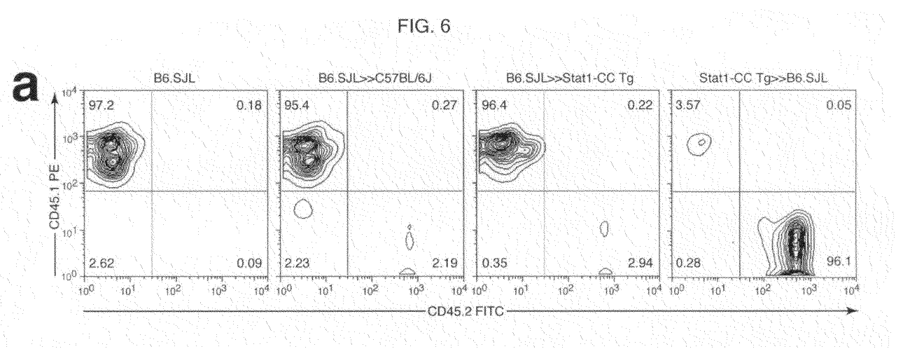

To address this issue, we generated chimeras by transferring bone marrow from wild-type B6.SJL mice (CD45.1) into irradiated Stat1-CC transgenic mice (CD45.2) or from Stat1-CC transgenic mice into irradiated wild-type B6.SJL mice. Engraftment was confirmed by flow cytometry analysis of CD45.1 versus CD45.2 alleles in peripheral blood leukocytes (FIG. 6a). Western blotting verified that the Stat1-CC transgene was expressed in peripheral blood leukocytes in wild-type B6.SJL mice reconstituted with bone marrow from Stat1-CC transgenic mice but was lost in Stat1-CC transgenic mice reconstituted with wild-type bone marrow (FIG. 6b). As we recently described for Stat1^{-/-} mice (Shornick, L. P., et al. 2008. J. Immunol. 180:3319-3328), this approach allowed us to dissect the role of Stat1-CC in the radiation-resistant compartment (especially host tissue cells) compared to the radiation-sensitive hematopoietic cells (especially immune cells).

In this setting, we found that Stat1-CC mice that received B6.SJL bone marrow retained resistance to EMCV infection whereas B6.SJL mice reconstituted with Stat1-CC bone marrow were still susceptible to infection with EMCV (FIG. 6c). Stat1-CC mice reconstituted with Stat1-CC bone marrow or C57BL/6J mice reconstituted with B6.SJL bone marrow were no different in their response to virus than Stat1-CC and B6.SJL mice, respectively. In these experiments, all mice were inoculated at 8 weeks after bone marrow transfer. At that stage, mice are 16-20 weeks of age and are able to survive longer than mice inoculated at 6-8 wk of age. Thus, death occurs at post-inoculation Day 12 in these older mice versus Day 4 found in younger mice.

The relative susceptibility of the bone marrow chimeras to EMCV infection correlated with the level of virus and consequent virus-induced damage in the tissue. Thus, viral levels were increased in C57BL/6 mice reconstituted with B6.SJL bone marrow or B6.SJL reconstituted with Stat1-CC bone marrow compared to Stat1-CC transgenic mice reconstituted with B6.SJL or Stat1-CC bone marrow (FIG. 6d). Here again, mice with higher viral levels also manifest increased tissue damage and inflammation (data not shown). Thus, the pattern of illness for B6.SJL mice reconstituted with Stat1-CC bone marrow was similar to wild-type mice as well as B6.SJL mice reconstituted with wild-type bone marrow. Moreover, the pattern of illness found in Stat1-CC transgenic mice that received B6.SJL bone marrow was similar to Stat1-CC transgenic mice as well as Stat1-CC transgenic mice that received Stat1-CC bone marrow. These results indicated a critical role for Stat1-CC in host tissue cells (e.g., pancreatic tissue cells) for controlling viral replication and thereby improving innate antiviral immunity.

### Stat1-CC Controls Viral Replication in U3A Cells.

As was the case for 2fTGH cells, we observed that the protective effects of Stat1-CC were evident with pretreatment of cultures with IFN-β or IFN-γ at high MOI and without pretreatment at low MOI (FIG. 5a,b). These findings show that Stat1-CC confers better viral clearance in U3A cells that are similarly engineered to express Stat1. These findings also indicate that IFN-β activation of Stat1-CC protects cells that are not yet infected with virus. Therefore, the protective effects of Stat1 can be more evident at lower MOI that allows for viral spread to neighboring cells. Furthermore, we found no benefit for viral clearance by expressing wild-type Stat1 in U3A cells. These results again indicate that endogenous levels of Stat1 do not limit the antiviral response, whereas Stat1-CC can enhance the cellular antiviral response. Without being limited by theory, the findings suggest that the enhancement of cellular response can result from more efficient activation of IFN-responsive genes.

In sum, our study of EMCV infection demonstrates that transgenic expression of a specifically modified Stat1 (designated Stat1-CC) can markedly increase the response to IFNs and improve the outcome from viral infection. The improved outcome relies on the capacity of Stat1-CC to suppress viral replication in host tissue cells and thereby decrease virus-induced tissue damage, inflammation, and morbidity during viral infection. These advantages during infection are unaccompanied by signs of toxicity under baseline conditions. Without being limited by theory, these observations likely result from the relative quiescence of the IFN-driven Stat1-dependent system in the absence of infection. Nonetheless, Stat1-CC activates a low level of enhanced IFN signaling at baseline that may be adequate to arm uninfected host cells and thereby prevent viral replication and spread. In contrast, in the present study, we found no additional protective effect of increasing Stat1 levels using retroviral transduction or transgene expression (FIG. 1, FIG. 4, FIG. 5).

### EXAMPLES

The following Examples are intended to be illustrative of various aspects of the present teachings and are not intended to be limiting of any claim. The methods and compositions described herein utilize laboratory techniques well known to skilled artisans, and can be found in laboratory manuals such as Sambrook, J., et al., Molecular Cloning: A Laboratory Manual, 3rd ed. Cold Spring Harbor Laboratory Press, Cold Spring Harbor, N.Y., 2001; Spector, D. L. et al., Cells: A Laboratory Manual, Cold Spring Harbor Laboratory Press, Cold Spring Harbor, N.Y., 1998; and Harlow, E., Using Antibodies: A Laboratory Manual, Cold Spring Harbor Laboratory Press, Cold Spring Harbor, N.Y., 1999. Pharmaceutical methods and compositions described herein, including methods for determination of therapeutically effective amounts, and terminology used to describe such methods and compositions, are well known to skilled artisans and can be adapted from standard references such as Remington: the Science and Practice of Pharmacy (Alfonso R. Gennaro ed. 19th ed. 1995); Hardman, J. G., et al., Goodman & Gilman's The Pharmacological Basis of Therapeutics, Ninth Edition, McGraw-Hill, 1996; and Rowe, R. C., et al., Handbook of Pharmaceutical Excipients, Fourth Edition, Pharmaceutical Press, 2003. As used in the description of the invention and the appended claims, the singular forms “a”, “an” and “the” are intended to include the plural forms as well, unless the context indicates otherwise. Experiments described herein may also involve the following materials and methods.

Transduced cells. U3A and 2fTGH cells were transduced with retroviral vectors MSCV-GFP, MSCV-Stat1-GFP, or MSCV-Stat1-CC-GFP as described previously (Zhang, Y., et al. 2005. J. Biol. Chem. 280:34306-34315). FACS purification resulted in a population of transduced cells that were >95% GFP-expressing.

Generation of transgenic mice. Wild-type C57BL/6J mice were from Jackson Laboratory. To generate transgenic mice, the pCAGGS vector that carries the CMV enhancer and chicken β-actin promoter (Niwa, H., et al. 1991. Gene 108:193-199) was used to generate pCAGGS-CMV-β-actin-Stat1-CC-3×Flag and pCAGGS-CMV-β-actin-Stat1-3×Flag. A SalI/PvuI-digested cDNA encoding CMV-β-actin-Stat1-CC-3×Flag or CMV-β-actin-Stat1-3×Flag was purified and microinjected into C57BL/6J zygotes. Transgenic founders were screened by PCR and then were bred to generate male mice for experiments. Transgene expression was assessed by Western blotting using mouse anti-Flag M2 mAb (Sigma). To assess IFN-responsiveness, wild-type and transgenic mice were treated with or without recombinant mouse IFN-γ or IFN-β (PBL Biomedical Laboratories) given by intraperitoneal injection at a dose of 20,000 or 200,000 units, respectively.

Western blot analysis. Cells were lysed and tissues were homogenized in 1% Nonidet P-40, 0.05M Tris, pH 8.0, 250 mM NaCl, 1 mM EDTA, containing 1 mM PMSF, 10 mg/ml aprotinin, 10 mg/ml leupeptin, 1 mM orthovanadate, 2 mM sodium pyrophosphate and 10 mM sodium fluoride. Cell and tissue extracts were subjected to SDS-PAGE, and proteins were blotted onto PVDF membrane (GE Healthcare) and incubated with anti-Flag antibody or rabbit anti-human phospho-Stat1 (Tyr-701) antibody (Cell Signaling Technology). Primary antibody binding was detected with sheep anti-rabbit IgG (Millipore) that was in turn detected by enhanced chemiluminescence.

RNA analysis. RNA was isolated using the RNeasy kit (QIAGEN), and mRNA levels were assessed using real-time PCR with the following forward and reverse primer pairs: 5′-AAGTGGAGCTCTCTGATCCTTCA-3′ (SEQ ID NO: 1) and 5′-GGCCTACCCCAGCAATGA-3′ (SEQ ID NO: 2) for Mx1; 5′-TGCTGCCCACCCAGTGA-3′ (SEQ ID NO: 3) and 5′-TGAGTGTGGTGCCTTTGC-3′ (SEQ ID NO: 4) for OAS; 5′-CGAGTGGACCTGAGGACCC-3′ (SEQ ID NO: 5) and 5′-AGTGTGAGAGCCGCCCTTG-3′ (SEQ ID NO: 6) for MHC Class I, 5′-CTACAGGTGTCACCCATGCC-3′ (SEQ ID NO: 7) and 5′GCTATCTTCCCTTCCTCATCC-3′ (SEQ ID NO: 8) for IRF-1; 5′CCTAAGATGACCTGCAGACGG-3′ (SEQ ID NO: 9) and 5′-TTTGACAGACTTCACCACCCC-3′ (SEQ ID NO: 10) for ICAM-1. All mRNA levels were normalized to levels of Gapdh mRNA using the TaqMan Rodent GAPDH Control Kit.

Viral inoculation and monitoring. Mouse encephalomyocarditis virus (EMCV, VR-129B) was obtained from ATCC and titered using a viral plaque-forming assay as described previously (Kimura, T., et al. 1994. Science 264:1921-1924). Mice were inoculated by intraperitoneal injection of EMCV at 1×10² or 1×10³ pfu in 100 μl PBS. Real-time PCR for EMCV-specific RNA was performed as described above using 5′-CTGCCTTCGGTGTCGC-3′ (SEQ ID NO: 11) and 5′-TGGGTCGAATCAAAGTTGGAG-3′ (SEQ ID NO: 12) as forward and reverse primers, respectively.

Immunohistochemistry. Immunostaining for 3×-Flag reporter was performed on paraffin-embedded heart tissue that was cut into 6-μm sections, blocked with 5% normal goat serum, and rabbit anti-Flag antibody (Sigma). Primary Ab binding was detected with biotinylated goat anti-rabbit antibody and the VECTASTAIN ABC-AP kit (Vector Laboratories). Sections were stained with an alkaline phosphatase red substrate and counterstained with hematoxylin. Immunostaining for EMCV was performed on tissue that was frozen, cut into 6-μm thick sections, fixed in cold acetone, blocked with 10% nonimmune goat serum, and incubated with mouse anti-EMCV RNA polymerase (3D protein) mAb from A.C. Palmenberg (Univ. Wisconsin) followed by peroxidase-conjugated goat anti-mouse IgG (Roche) and exposure to 3-amino-9-ethylcarbazole (Sigma). For histopathological studies, mouse organs were fixed in 10% formalin, embedded in paraffin, and stained with hematoxylin and eosin.

Bone marrow transfer. Bone marrow transfer was performed as described previously (18). For the present experiments, 1×10⁷ bone marrow cells were used to reconstitute lethally irradiated (9.5 to 10 Gy) recipient mice. Chimeric mice were analyzed at 8 wk after bone marrow transfer, and bone marrow engraftment was assessed by flow cytometry of peripheral blood leukocytes (PBLs). Single cell PBL suspensions were stained with PE-conjugated mouse anti-CD45.1 and FITC-conjugated mouse anti-CD45.2 (BD Biosciences) for 30 min at 4° C. Data acquisition was performed using a BD FACSCalibur flow cytometer interfaced to CellQuest (BD Biosciences) and FlowJo software (version 6.4.7, Tree Star, Inc.).

Statistical analysis. Mouse survival was assessed by Kaplan-Meier analysis. Values for real-time PCR and viral titer were analyzed using paired t-test. Significance level for all analyses was p value<0.05. All values represent mean±SEM.

### Example 1

This Example illustrates improved control of viral replication in Stat1-CC-expressing 2fTGH human cells.

These experiments are illustrated in FIG. 1. In these experiments, as shown in FIG. 1a, 2fTGH cells were transduced with MSCV2.2 retroviral vector encoding GFP, Stat1-GFP, or Stat1-CC-GFP and then were treated with IFN-γ (100 U) or IFN-β (1000 U/ml). Cell lysates were Western blotted using anti-phospho-Stat1 (Tyr701) or Stat1 antibody. In FIG. 1b, 2fTGH cells expressing GFP, Stat1-GFP, or Stat1-CC-GFP were inoculated with EMCV (MOI 1 for 24 h) without (NT) or with pre-incubation with IFN-γ (100 U/ml) or IFN-β (10 or 1000 U/ml) for 6 h, and EMCV-specific RNA levels were assessed on post-inoculation (PI) Day 2. In FIG. 1c, for conditions in FIG. 1b, cells were also inoculated with indicated MOI. * indicates a significant difference from corresponding value for 2fTGH cells transduced with vector-GFP control.

The data demonstrate that expression of Stat1-CC confers better viral clearance in U3A parental 2fTGH cells that contain endogenous Stat1 (FIG. 1a,b). These findings suggested that IFN-β activation of Stat1-CC protects cells that are not yet infected with virus. Therefore, the protective effects of Stat1 may be more evident at lower MOI that allows for viral spread to neighboring cells. Indeed, we found a significant decrease in viral replication rates in Stat1-CC-expressing cells compared to Stat1-expressing or native 2fTGH cells (FIG. 1c). Furthermore, found no benefit for viral clearance by expressing wild-type Stat1 in 2fTGH cells. These results indicate that endogenous levels of Stat1 do not limit the antiviral response, whereas Stat1-CC, by providing more efficient activation of IFN-responsive genes, can improve the antiviral response. Without being limited by theory, the present inventor believes that enhanced cellular response can be a result of more efficient activation of IFN-responsive genes compared to Stat1 in cells expressing Stat1-CC.

### Example 2

This Example illustrates Stat1-CC transgene expression and activation in mice.

These experiments are illustrated in FIG. 2, as follows. FIG. 2a: Western blots of tissue homogenates from WT, Stat1 transgenic, and Stat1-CC transgenic mice using anti-Flag or anti-β-actin antibody. FIG. 2b: Western blots of myocardial tissue homogenates from WT, Stat1 transgenic, and Stat1-CC transgenic mice that were untreated or treated with IFN-γ (20,000 U given intraperitoneally) using anti-phospho-Stat1 (Tyr701), Stat1, or anti-Flag antibody. FIG. 2c: Representative photomicrographs of myocardial tissue from WT, Stat1 transgenic, and Stat1-CC transgenic mice treated with IFN-γ as described in FIG. 2b. Sections were stained using anti-Flag antibody and an alkaline phosphatase system and then counterstained with hematoxylin. Control staining with non-immune IgG gave no signal above background (data not shown).

In these experiments, we generated Western blots of tissue homogenates from WT, Stat1 transgenic, and Stat1-CC-transgenic mice using anti-Flag or anti-β-actin antibody as shown in FIG. 2a. As expected from previous use of this promoter system, we found high-level transgene expression in heart, pancreas, and skeletal muscle tissues, and intermediate-level expression in brain, lung, thymus, and spleen (FIG. 2a). We also performed Western blots of myocardial tissue homogenates from WT, Stat1 Transgenic, and Stat1-CC Transgenic mice that were untreated or treated with IFN-γ. As shown in FIG. 2b, lysates were Western blotted using anti-phospho-Stat1 (Tyr701), Stat1, or anti-Flag antibody. As shown in FIG. 2b, we also obtained representative photomicrographs of myocardial tissue from WT, Stat1 Transgenic, and Stat1-CC Transgenic mice treated with IFN-γ. Sections were stained using anti-Flag Ab and an alkaline phosphatase system and then counterstained with hematoxylin. These data show that the Stat1-CC transgene product also exhibited prolonged Tyr-701 phosphorylation and nuclear localization in response to IFN-γ treatment compared to wild-type Stat1. The results are similar to behavior in Stat1-CC-expressing cell lines.

### Example 3

This Example illustrates enhanced IFN efficacy for gene expression in Stat1-CC transgenic mice.

These experiments are illustrated in FIG. 3, as follows. FIG. 3a: Schematic representation of the expression cassette used for generating Stat1- and Stat1-CC transgenic mice. FIG. 3b: Real-time PCR analysis of Stat1-CC-dependent target mRNA levels in pancreas from WT, Stat1 transgenic, and Stat1-CC transgenic mice at baseline and 1 day after treatment with IFN-β. * indicates a significant difference from corresponding Stat1 transgenic control mice.

In these experiments, Stat1 and Stat1-CC transgenic mice were generated as described in Example 2 and using expression cassettes shown in FIG. 3a. We performed real-time PCR analysis of Stat1-CC-dependent target mRNA levels in pancreas from WT, Stat1 Transgenic, and Stat1-CC Transgenic mice at baseline and 1 day after treatment with IFN-β as shown in FIG. 3b. This data show that Stat1-CC transgenic mice exhibit increased gene expression in response to injected IFN-γ and IFN-β, indicating that Stat1-CC conferred increases in IFN efficacy in vivo similar to those found in vitro. These data also shows marked increases in baseline gene expression without IFN treatment. Because Stat1-CC requires ligand-dependent phosphorylation for function (Zhang, Y., et al. 2005. J. Biol. Chem. 280:34306-34315), these findings indicate that low-level production of type I IFN is able to drive Stat1-CC activation and consequent increases in gene expression in vivo even under baseline conditions. As further developed below, Stat1-CC transgenic mice exhibited an expression profile that could be broadly grouped into IFN-responsive genes that contribute to antiviral defense directly through the innate immune response (especially by inhibition of viral replication) and indirectly through the adaptive immune response (especially by antigen processing and presentation).

### Example 4

This Example illustrates protection against viral infection in Stat1-CC transgenic mice.

These experiments are illustrated in FIG. 4, as follows. FIG. 4a: Wild-type (WT) and CMV-b-actin-Stat1 and Stat1-CC transgenic mice were inoculated with EMCV (100 pfu) and monitored for survival by Kaplan-Meier analysis (n=15-27 per group). FIG. 4b: EMCV-specific RNA levels in mouse heart, brain and pancreas from conditions in FIG. 4a. * indicates a significant decrease from corresponding WT. FIG. 4c: Immunostaining in pancreatic tissues reveals decreased levels of EMCV in Stat1-CC transgenic mice. FIG. 4d: pancreas sections of mice from FIG. 4a revealing EMCV tissue damage in concert with sites of viral replication.

In these experiments, wild type, Stat1 and Stat1-CC transgenic mice were inoculated with EMCV or an equivalent amount of UV-inactivated EMCV and monitored for survival by Kaplan-Meier analysis. As shown in FIG. 4a, inoculation with EMCV at 100 pfu caused a lethal infection in wild-type C57BL/6J mice as well as Stat1 transgenic mice. By contrast, Stat1-CC transgenic mice were markedly protected from viral infection: Stat1-CC transgenic mice with the same genetic background survived at a rate of 97% at this viral inoculum and at a rate of 82% even at 100-fold higher inoculum. At lower viral inoculum of 3 pfu, wild-type and Stat1 transgenic mice survived at a rate of 25-28% whereas Stat1-CC mice survived at a rate of 100%. Furthermore, as shown in FIG. 4b, improved survival rate was associated with a marked decrease in viral titers in heart, brain, and pancreas in Stat1-CC transgenic mice. In these experiments, EMCV-specific RNA levels in mouse heart, brain, and pancreas from conditions in FIG. 4a were determined using real-time PCR. Values represent mean±SEM (n=5-8 mice per group). * indicates a significant decrease from corresponding WT control value. In addition, immunostaining in pancreatic tissues revealed decreased levels of EMCV in Stat1-CC transgenic mice compared to wild-type or Stat1 transgenic mice (FIG. 4c). In these experiments, representative photomicrographs of pancreas sections of mice from FIG. 4a were immunostained with anti-EMCV mAb and counterstained with hematoxylin. In FIG. 4d, representative photomicrographs of pancreas sections of mice from FIG. 4a stained with hematoxylin and eosin were analyzed in a necropsy investigation. These sections reveal that EMCV tissue damage occurred in concert with the sites of viral replication. The major site of injury appeared to be the pancreas (where we found the highest viral titers), followed by brain and heart. Tissue sections showed severe edema, damage, and inflammatory cell infiltration in wild-type and Stat1 transgenic mice after EMCV infection (FIG. 4d). By contrast, pancreas tissue exhibited only little of these abnormalities in Stat1-CC transgenic mice infected with EMCV. The major site of damage to the pancreas was localized to exocrine tissue, with relative sparing of islet tissue.

### Example 5

This Example illustrates protection against EMCV infection and consequent encephalitis and myocarditis in Stat1-CC transgenic mice.

These experiments are illustrated in FIG. 5, as follows. FIG. 5a: WT mice and CMV-β-actin-Stat1 and Stat1-CC transgenic mice were inoculated with EMCV (3 and 10,000 pfu) and monitored for survival by Kaplan-Meier analysis (n=15-27 per group). FIG. 5b: Representative photomicrographs of brain tissue sections were obtained from mice on PI Day 0 and Day 4 and were stained with hematoxylin and eosin. FIG. 5c: Corresponding photomicrographs of myocardial tissue sections.

In these experiments, similar to the case for pancreas, we found a marked decrease in encephalitis in Stat1-CC transgenic mice after EMCV infection. Thus, we found neuronal shrinkage and necrosis in the brains of wild-type and Stat1 transgenic mice, whereas these pathological alterations were not observed in Stat1-CC transgenic mice (FIG. 5b). We also detected the development of a dilated cardiomyopathy based on gross pathology at necropsy as well as echocardiography in a subgroup of wild-type and Stat1 transgenic mice (data not shown).

In addition, we found mild inflammation and edema in myocardial tissue in wild-type or transgenic mice after EMCV infection (FIG. 5c). The observed changes in myocardial function were therefore most likely due to toxicity of the infection as well as a low level of viral replication at this site. These abnormalities in myocardial function and histology were not detected in Stat1-CC transgenic mice infected with EMCV. Taken together, the findings indicate that expression of the Stat1-CC transgene allowed the host to achieve lower levels of virus and virus-induced tissue damage in brain and heart as well as pancreas.

### Example 6

This Example illustrates the effect of bone marrow transfer on susceptibility to EMCV infection and that Stat1-CC controls viral replication at the tissue host cell level.

These experiments are illustrated in FIG. 6, as follows. FIG. 6a: Flow cytometry confirming engraftment. FIG. 6b: Western blotting verifying expression of Stat1-CC transgene in peripheral blood leukocytes in wild-type B6.SJL mice reconstituted with bone marrow from Stat1-CC transgenic mice but lost in Stat1-CC transgenic mice reconstituted with wild-type bone marrow. FIG. 6c: Recipient WT B6.SJL, WT C57BL/6J, or CMV-b-actin-Stat1-CC Transgenic mice were reconstituted with WT B6.SJL or Stat1-CC Transgenic donor bone marrow cells. Eight weeks later, chimeric mice were inoculated with EMCV (100 pfu) and monitored for survival by Kaplan-Meier analysis (n=15 per group). * indicates a significant increase in survival compared to WT consisting of B6.SJL bone marrow transfer into C57BL6/J (SJL>>B6). FIG. 6d: Corresponding analysis of EMCV levels in pancreas from PI Day 4 for each group of mice in (a). * indicates a significant decrease from WT control (SJL>>B6).

In these experiments, chimeras were generated by transferring bone marrow from wild-type B6.SJL mice (CD45.1) into irradiated Stat1-CC transgenic mice (CD45.2) or from Stat1-CC transgenic mice into irradiated wild-type B6.SJL mice. As shown in FIG. 6a, recipient WT B6.SJL, WT C57BL/6J, or Stat1-CC Transgenic mice were lethally irradiated and reconstituted with WT B6.SJL or Stat1-CC transgenic donor bone marrow cells (1×10⁷ cells/mouse). Engraftment was confirmed by flow cytometry analysis of CD45.1 versus CD45.2 alleles in peripheral blood leukocytes (FIG. 6a). Eight weeks after reconstitution, peripheral blood leukocytes (PBLs) from chimeric mice were analyzed by flow cytometry using PE-conjugated anti-CD45.1 mAb and FITC-conjugated anti-CD45.2 mAb. Western blotting verified that the Stat1-CC transgene was expressed in peripheral blood leukocytes in wild-type B6.SJL mice reconstituted with bone marrow from Stat1-CC transgenic mice but was lost in Stat1-CC transgenic mice reconstituted with wild-type bone marrow. As shown in FIG. 6b, Stat1-CC transgene expression was investigated in WT, Stat1-CC Transgenic, and chimeric mice using Western blot analysis of PBL lysates against anti-Flag Ab followed by anti-β-actin Ab. Eight weeks after reconstitution, chimeric mice were also inoculated with EMCV (100 pfu) and monitored for survival by Kaplan-Meier analysis (n=15 per group). * indicates a significant increase in survival compared to WT control consisting of B6.SJL bone marrow transfer into C57BL6/J (SJL>>B6). Stat1-CC mice that received B6.SJL bone marrow retained resistance to EMCV infection whereas B6.SJL mice reconstituted with Stat1-CC bone marrow were still susceptible to infection with EMCV (FIG. 5c). Stat1-CC mice reconstituted with Stat1-CC bone marrow or C57BL/6J mice reconstituted with B6.SJL bone marrow were no different in their response to virus than Stat1-CC and B6.SJL mice, respectively. In these experiments, all mice were inoculated at 8 weeks after bone marrow transfer. At that stage, mice are 16-20 weeks of age and are able to survive longer than mice inoculated at 6-8 wk of age. The data indicates that death occurs at post-inoculation Day 12 in these older mice versus Day 4 found in younger mice. FIG. 6d shows that the relative susceptibility of the bone marrow chimeras to EMCV infection correlates with the level of virus and consequent damage in the tissue. In these experiments, C57BL/6 mice reconstituted with B6.SJL bone marrow or B6.SJL reconstituted with Stat1-CC showed much higher viral RNA levels, whereas Stat1-CC transgenic mice reconstituted with B6.SJL bone marrow or Stat1-CC had much lower or undetectable viral RNA levels. FIG. 6d shows a corresponding analysis of EMCV-specific RNA in pancreas from PI Day 4 for each group of mice in FIG. 6c. * indicates a significant decrease from WT control (SJL>>B6). From these and other data, we conclude that mice with higher viral levels also manifest increased tissue damage. Thus, the pattern of illness for B6.SJL mice reconstituted with Stat1-CC bone marrow was similar to wild-type mice as well as B6.SJL mice reconstituted with wild-type bone marrow. Moreover, the pattern of illness found in Stat1-CC transgenic mice that received B6.SJL bone marrow was similar to Stat1-CC transgenic mice as well as Stat1-CC transgenic mice that received Stat1-CC bone marrow, indicating a critical role for Stat1-CC in host tissue cells (e.g., pancreatic tissue cells) for controlling viral replication and thereby improving innate antiviral immunity.

### Example 7

This example illustrates enhanced IFN-dependent gene expression in Stat1-CC-expressing human U3A cells.

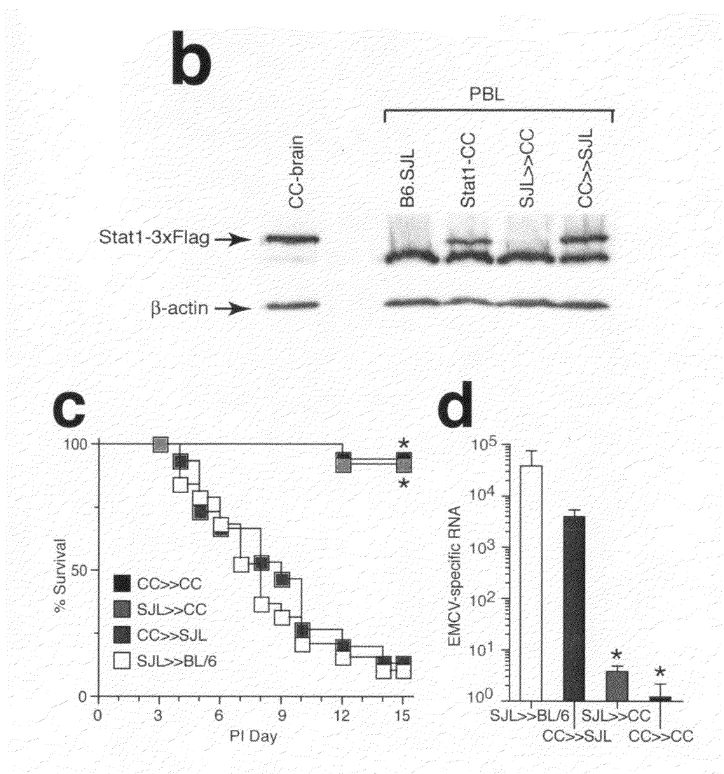

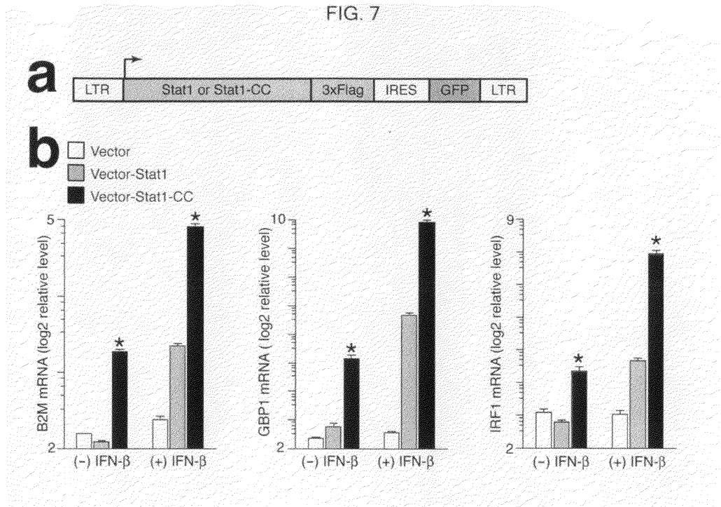

These experiments are illustrated in FIG. 7, as follows. FIG. 7a: Schematic representation of the expression cassette used for generating Stat1- and Stat1-CC-expressing U3A cells. FIG. 7b: Real-time PCR analysis Stat1-CC-dependent target mRNA levels in U3A cells expressing GFP alone, Stat1, or Stat1-CC at baseline and I day after treatment with IFN-β. * indicates a significant difference from corresponding Stat1-expressing cells.

In these experiments, we generated Stat1- and Stat1-CC-expressing U3A cells using the expression cassette shown in FIG. 7a. We used real-time PCR analysis to determine Stat1-CC-dependent target mRNA levels in U3A cells expressing GFP alone, Stat1, or Stat1-CC at baseline and 1 day after treatment with IFN-β. The findings show an increase in target gene expression in Stat1-CC-expressing cells under baseline conditions (without IFN treatment) and under IFN treatment conditions.

### Example 8

This example illustrates enhanced control of viral replication in Stat1-CC-expressing human U3A cells.

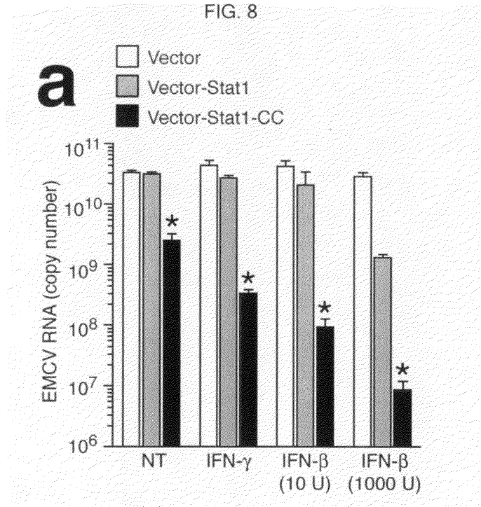

These experiments are illustrated in FIG. 8, as follows. FIG. 8a: Control as well as Stat1- and Stat1-CC-expressing U3A cells were inoculated with EMCV (MOI 1 for 24 h) without (NT) or with pre-incubation with IFN-γ (100 U/ml) or IFN-β (10 or 1000 U/ml) for 6 h, and virus-specific RNA levels were assessed by real-time PCR on PI Day 2. FIG. 8b: For NT conditions in FIG. 8a, cells were inoculated with indicated EMCV inoculum for 24 h, and viral RNA levels were determined as in FIG. 8a.

We previously reported improved antiviral action in Stat1-CC-versus Stat1-expressing or Stat1-null U3A cells that are pre-treated with IFN and then infected with EMCV (ref. (23) and FIG. 8a). Here we show that expression of Stat1-CC also confers an improvement in defense against EMCV in U3A cells that increases further at lower inoculums in the absence of IFN treatment. As shown in FIG. 8b, control as well as Stat1- and Stat1-CC-expressing U3A cells were inoculated with EMCV (MOI 1 for 24 h) without (NT) or with pre-incubation with IFN-γ (100 U/ml) or IFN-β (10 or 1000 U/ml) for 6 h, and virus-specific RNA levels were assessed by real-time PCR on PI Day 2. (b) For NT conditions in (a), cells were inoculated with indicated EMCV inoculum for 24 h.

### Example 9

This example illustrates protection against IAV infection in b-actin-CMV-Stat1-CC transgenic mice.

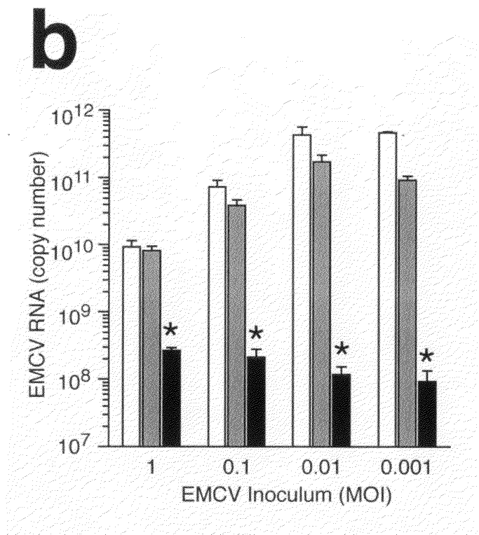

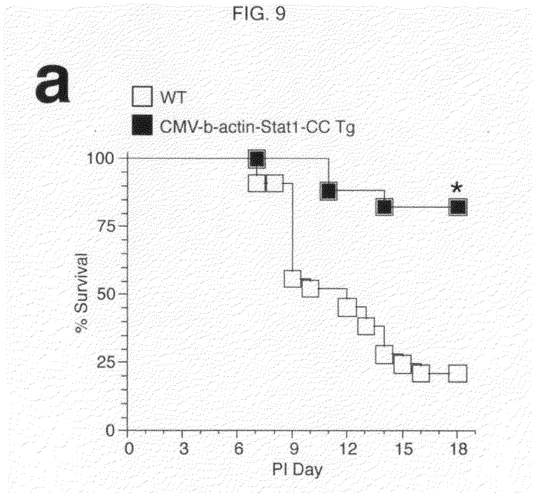

These experiments are illustrated in FIG. 9, as follows. FIG. 9a: WT and CMV-β-actin Transgenic mice were inoculated with influenza A virus (IAV-H1N1, 25 pfu) and monitored for survival by Kaplan-Meier analysis (n=17-22 mice per group). FIG. 9b: Corresponding IAV-specific RNA levels in lung were determined using real-time PCR. Values represent mean±SEM (n=5-8 mice per group). * indicates a significant decrease from corresponding WT control value. FIG. 9c: For conditions in FIG. 9a, corresponding lung sections were immunostained with rat anti-mouse neutrophil Ab. Bar=20 μm. FIG. 9d: For conditions in FIG. 9a, mice were analyzed for BAL fluid cell counts. All values represent mean±SEM, and * indicates a significant difference from corresponding WT control.

In these experiments, we found that CMV-b-actin-Stat1-CC transgenic mice were also protected against infection with influenza A virus (IAV-H1N1 type). For example, Stat1-CC transgenic mice survival was 82% compared to 20% for wild-type mice (FIG. 9a). Viral titers showed a marked decrease in Stat1-CC transgenic mice compared to wild-type mice, and concomitant protection against neutrophilic inflammation and tissue damage (FIG. 9b, FIG. 9c, and data not shown).

### Example 10

This example illustrates protection against IAV infection in rCCSP-Stat1-CC transgenic mice.

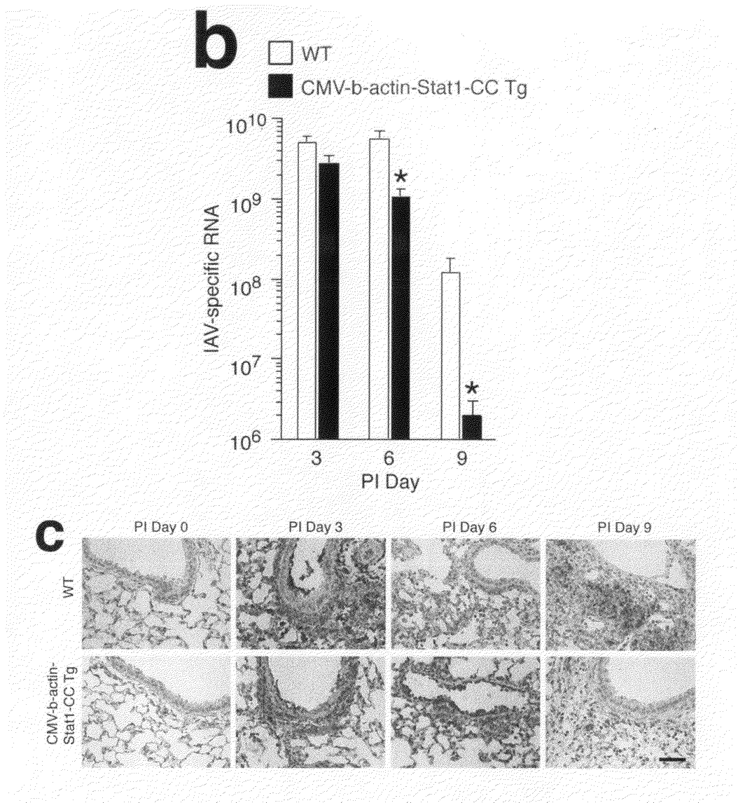

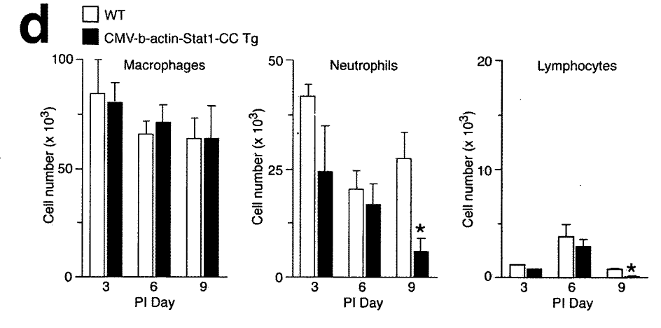

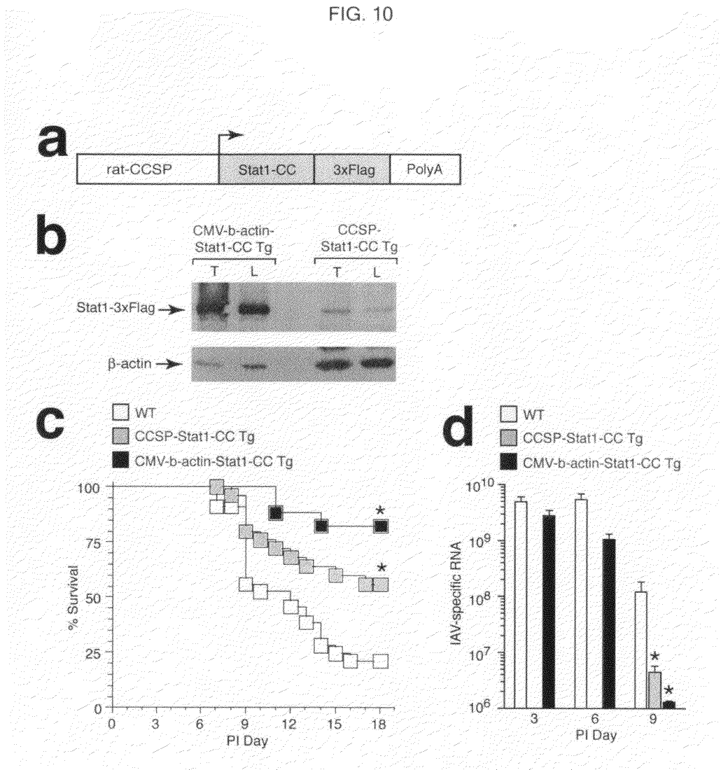

These experiments are illustrated in FIG. 10, as follows. FIG. 10a: Schematic representation of the expression cassette used for generating Stat1-CC transgenic (Transgenic) mice using the rat CCSP gene promoter. FIG. 10b: Western blot analysis of tracheal (T) and lung (L) tissue lysates from (WT) C57BL/6J control mice versus rCCSP-Stat1-CC using anti-3×Flag Ab or control anti-β-actin Ab. FIG. 10c: WT control and CMV-b-actin-Stat1-CC and rat CCSP-Stat1-CC Transgenic mice were inoculated with influenza A virus (IAV-H1N1, 25 pfu) and monitored for survival by Kaplan-Meier analysis (n=17-22 per group). FIG. 10d: Corresponding IAV-specific RNA levels in lung were determined using real-time PCR. Values represent mean±SEM (n=5-8 mice per group). * indicates a significant decrease from corresponding WT control value.

In these experiments, we further addressed the issue of Stat1-CC site of action for protection against influenza virus. In the first set of experiments, we generated transgenic mice using a cell-type specific promoter (based on the rat CCSP gene promoter) that directs gene expression to a subset of airway epithelial cells (predominantly Clara cells) as described previously (Perl, A.-K. T. et al, 2005. Am. J. Respir. Cell Mol. Biol. 33:455-462) and illustrated in FIG. 10a. Transgene expression in mouse trachea and lung was confirmed by Western blot analysis of these tissues, although expression was significantly decreased compared to the CMV-b-actin system (FIG. 10b). Despite achieving suboptimal expression levels in only a subset of airway epithelial cells, we still found that rCCSP-Stat1-CC transgenic mice showed a significant benefit, since they exhibited 60% survival compared to 20% for wild-type control mice after inoculation with IAV-H1N1 (FIG. 10c). In concert with improved survival, rCCSP-Stat1-CC transgenic mice also showed decreased weight loss and decreased levels of IAV-H1N1 in lung tissue compared to wild-type mice (FIG. 10d and data not shown). These initial results demonstrate that expression of Stat1-CC in airway epithelial cells efficiently controls viral replication and increases the rate of survival for an important human pathogen. However, we would expect even better protection if transgene expression were also directed to ciliated airway epithelial cells, which are a primary host cell for IAV replication (Ibricevic, A. et al., 2006. J. Virol. 80:7469-7480) and unpublished observations, Y Zhang and M J Holtzman. In that regard, we have recently described the first transgenic promoter system to selectively target ciliated epithelial cells (Zhang, Y. et al., 2006. Am. J. Respir. Cell Mol. Biol. 36:515-519).

### Example 11

This example illustrates protection against IAV infection after AAV-mediated Stat1-CC gene transfer in mice.

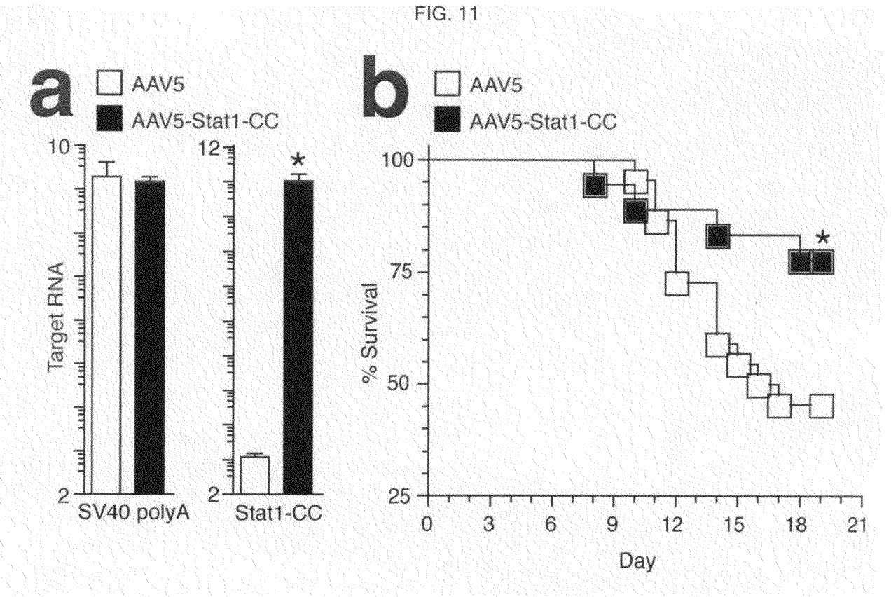

These experiments are illustrated in FIG. 11, as follows. FIG. 11a: WT mice were treated with AAV5 or AAV5-Stat1-CC (3×10¹⁰ particles on day 0 and day 2), and lung levels of AAV5 (using SV40 polyA as a marker) and Stat1-CC mRNA were determined at post-AAV5-treatment (PAT) Day 21. FIG. 11b: AAV5 or AAV5-Stat1-CC-treated mice were inoculated with IAV (25 pfu) at PAT Day 21, and monitored for survival by Kaplan-Meier analysis (n=18-22 mice per group). * indicates a significant increase from AAV5 control.

In these experiments, we used a gene transfer system with an adeno-associated virus serotype 5 (AAV5) vector (Patel, A. C., et al., 2006. Physiol. Genomics 25:502-513). For these experiments, mice are treated with AAV5-Stat1-CC or control AAV5 delivered intranasally in the same manner as for viral inoculations. By three weeks after treatment, this method of gene transfer achieves a marked increase in the lung levels of Stat1-CC, and this level is sustained for at least another 7 weeks (Patel, A. C., et al., 2006. Physiol. Genomics 25:502-513 and FIG. 11a). Using this approach, we found that mice treated with AAV5-Stat1-CC exhibited a survival rate of 80% after infection with IAV-H1N1 compared to a rate of 45% in mice treated with AAV5 control vector (FIG. 11b). The improved survival rates for AAV5-Stat1-CC-treated mice were therefore similar to CMV-b-actin-Stat1-CC transgenic mice after IAV-H1N1 infection, as was the concomitant decrease in lung levels of IAV-H1N1 (data not shown). Together, these data support our efforts to use AAV5-mediated gene transfer of Stat1-CC to the airway epithelium as a therapeutic strategy for respiratory viral infection.

### Example 12

This example illustrates decreased IAV levels in Stat1-CC-expressing U3A human cells.

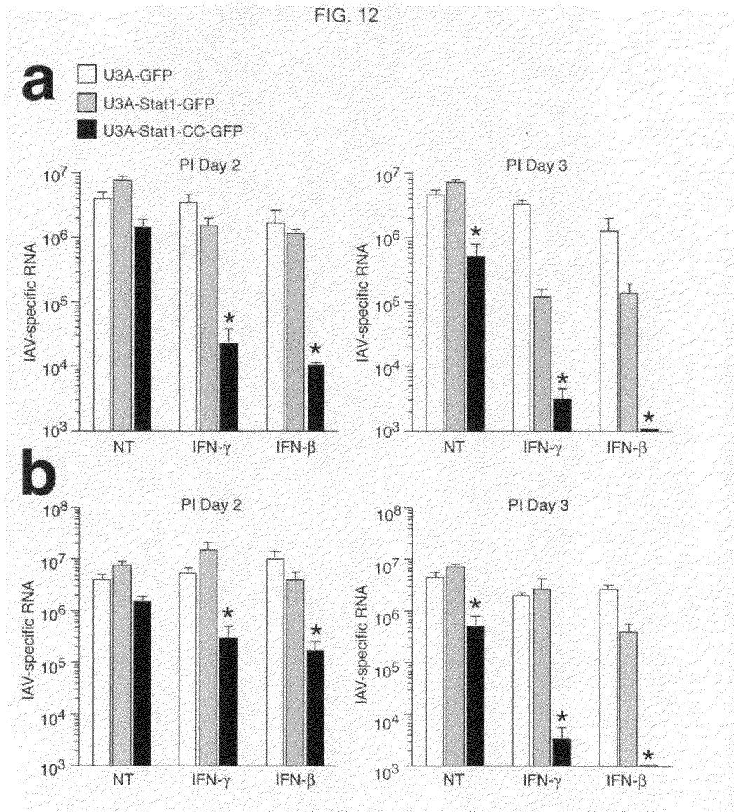

These experiments are illustrated in FIG. 12, as follows. FIG. 12a: U3A cells transduced with MSCV-GFP, MSCV-Stat1 or MSCV-Stat1-CC were pre-incubated without or with IFN-γ (100 U/ml) or IFN-β (1000 U/ml) for 6 h and then inoculated with IAV-H1N1 (MOI 1). Cell supernatants were analyzed for virus-specific RNA at post-inoculation (PI) Day 2 and 3. FIG. 12b: Same conditions were used as in FIG. 12a, except that cells were first inoculated with influenza (MOI 1), and IFN-γ or IFN-β was added 6 h later. All values represent mean±SEM (n=3), and * indicates a significant decrease from U3A-Stat1-GFP cells.

In these experiments, we show that expression of Stat1-CC confers an improvement in defense against IAV in U3A cells (FIG. 12a and data not shown), and that Stat1-CC is effective even when IFN is delivered after inoculation (FIG. 12b).

### Example 13

This example illustrates that Stat1-CC transgene protects against SeV infection.

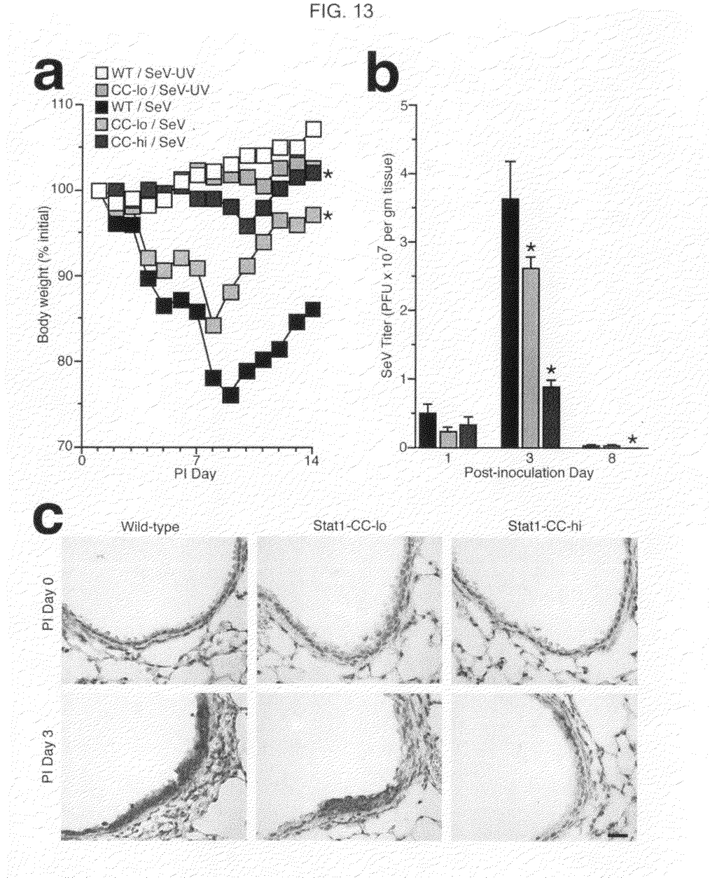

These experiments are illustrated in FIG. 13, as follows. FIG. 13a: WT and CMV-β-actin-Stat1-CC transgenic mice (high and low expressers) were inoculated with SeV (1×10⁵ pfu) or UV-inactivated SeV and monitored for body weight (n=15 mice per group). * indicates a significant difference from SeV-inoculated WT mice. FIG. 13b: Plaque-forming assay of SeV titers in mouse lungs from FIG. 13a. * indicates a significant decrease from SeV-inoculated WT mice. FIG. 13c: Representative lung sections from FIG. 13a immunostained for SeV.

In these experiments, we found that CMV-b-actin-Stat1-CC mice are resistant to infection with Sendai virus (SeV). In this case, we showed that CMV-b-actin-Stat1-CC transgenic mice with high-level Stat1-CC expression were protected more effectively compare to a second transgenic line with lower expression of Stat1-CC (FIG. 13a). Protection against SeV occurred in concert with decreased lung levels of SeV (FIG. 13b,c).

### Example 14

This example illustrates that Stat1-CC transgene protects against chronic inflammatory lung disease after viral infection.

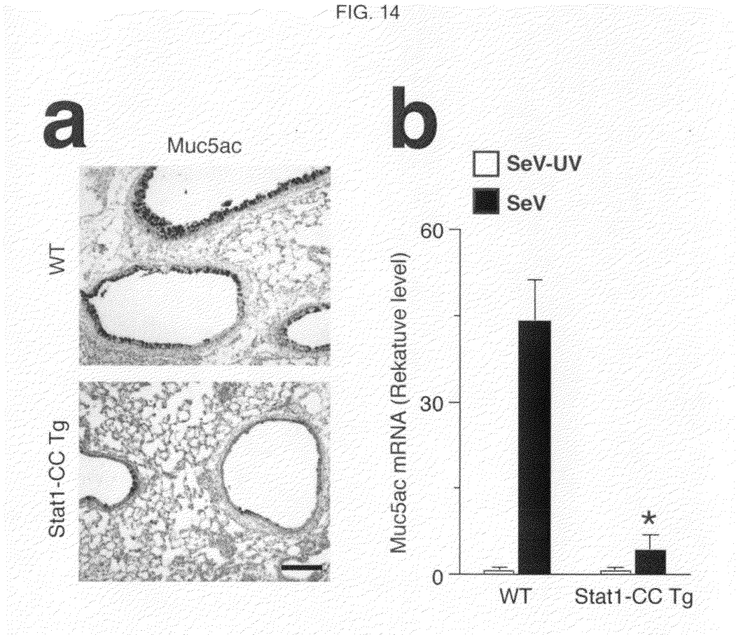

These experiments are illustrated in FIG. 14, as follows. FIG. 14a: WT and CMV-β-actin-Stat1-CC transgenic mice were inoculated with SeV or SeV-UV and corresponding lung sections were immunostained for MUC5AC at PI Day 49. FIG. 14b: Quantification of results from FIG. 14a. * indicates a significant decrease from corresponding WT mice.

In these experiments, we show that Stat1-CC transgenic mice are protected against the subsequent development of chronic inflammatory lung disease. For example, wild-type C57BL/6J mice develop chronic inflammatory lung disease manifested by mucous cell metaplasia (Tyner, J. W., et al., 2005. Nat. Med. 11:1180-1187; Patel, A. C. et al., 2006. Physiol. Genomics 25:502-513; Grayson, M. H., et al., 2007. J. Exp. Med. 204:2759-2769; Kim, E. Y., et al. 2008. Nat. Med. 14; Walter, M. J., et al. 2002. J. Clin. Invest. 110:165-175). However, Stat1-CC transgenic mice exhibited nearly complete blockade of chronic mucous cell metaplasia after SeV infection (FIG. 14).

### Example 15

This Example illustrates the capacity of Stat1-CC to increase IFN-induced apoptosis in U3A human cells.

These experiments present a flow cytometric analysis of U3A cells expressing GFP, Stat1 and GFP, or Stat1-CC and GFP transgenes without and with treatment with IFN-γ (100 U/ml) or IFN-β (1000 U/ml) for 24 h in absence or presence of zVAD. In these experiments, cell viability was based on propidium iodide exclusion and cell side-scatter. Values represent mean±SEM (n=9). * indicates a significant decrease from the value for Stat1-expressing cells.

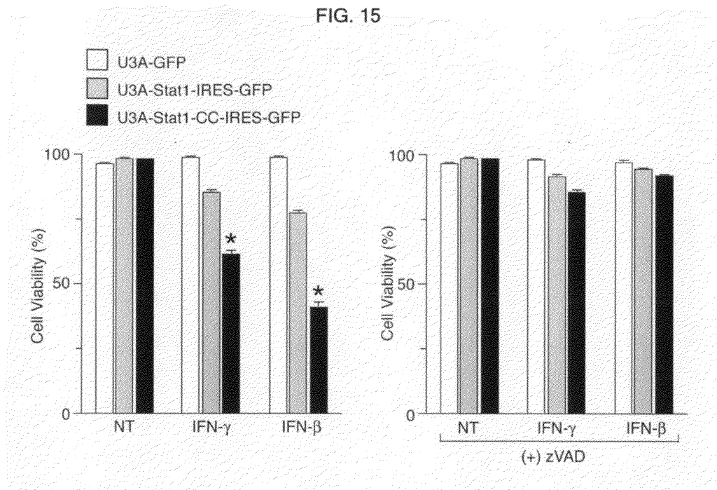

In these experiments, retroviral-mediated gene transfer to Stat1-deficient U3A cells established stable cell lines for expression of wild-type and mutant Stat1-CC. Transduced U3A cells were then analyzed for cell viability with and without treatment with IFN-β or IFN-γ. The level of IFN-induced cell death was significantly increased in Stat1-CC-expressing U3A cells compared to Stat1-expressing cells (FIG. 15). Under these conditions, the cell death was inhibited by treatment with the caspase inhibitor zVAD, indicating that the death was due to apoptosis (programmed cell death).

### Example 16

This Example illustrates the capacity of Stat1-CC to inhibit tumor formation in vivo.

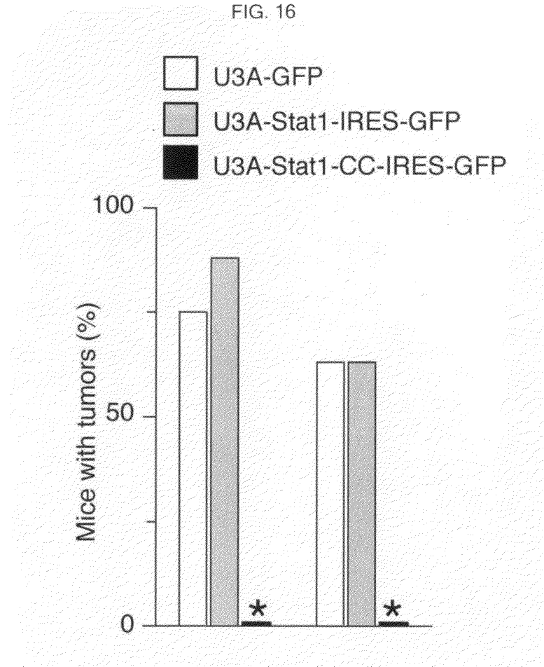

As illustrated in FIG. 16, on protocol day 0, RAG1-null mice were injected subcutaneously with 2×10⁶ U3A cells and then were treated without or with human recombinant IFN-β (10,000 U injected at the site of inoculated cells on days 3, 5, 7, 9, 11, 13, and 15). Tumor formation was determined on day 21. Values represent mean for 4-8 mice per group. * indicates a significant decrease from the value for Stat1-expressing cells.

For these experiments, transduced U3A cells were injected into mouse skin and assessed for tumor formation in the presence or absence of treatment with IFN-β, as set forth in Table I. We found that tumor formation was significantly inhibited after infection of Stat1-CC-expressing U3A cells compared to Stat1-expressing cells either with or without IFN-β treatment (FIG. 16). These findings indicated that Stat1-CC inhibited tumor formation in vivo, and that native levels of endogenous IFN-β were sufficient to drive the effectiveness of Stat1-CC in promoting tumor cell death and thereby preventing tumor formation.

TABLE I
This table provides the specific data for Example 16. On protocol
day 0, RAG1-null mice were injected subcutaneously with
2 × 10⁶ U3A cells and then were treated without or with
human IFN-β (10,000 U injected at the site of inoculated cells on
days 3, 5, 7, 9, 11, 13, and 15). On day 21, tumor size was measured
with a scale.

| | Tumor Cell Type | | |
|---|---|---|---|
| Treatment | U3A-GFP | U3A-Stat1 | U3A-Stat1-CC |
| (−) IFN-β | 6/8 | 7/8 | 0/4 |
| (+) IFN-β | 5/8 | 5/8 | 0/8 |

The present disclosure includes the following aspects.
1. A method of treating a viral infection, comprising administering to a subject a vector comprising a Stat1-CC transgene.
2. A method of treating a viral infection in accordance with aspect 1, wherein the vector is an adeno-associated virus (AAV).
3. A method of treating a viral infection in accordance with aspect 2, wherein the AAV is an AAV5.
4. A method of treating a viral infection in accordance with aspect 1, wherein the vector further comprises a promoter operably linked to the Stat1-CC transgene.
5. A method of treating a viral infection in accordance with aspect 4, wherein the promoter operably linked to the Stat1-CC transgene is a CMV-β-actin promoter.
6. A method of treating a viral infection in accordance with aspect 4, wherein following the administration of the vector, the subject comprises one or more cells which express the Stat1-CC transgene.
7. A method of treating a viral infection in accordance with aspect 1, wherein the viral infection is of a virus which induces a cellular interferon response.
8. A method of treating a viral infection in accordance with aspect 7, wherein the virus is selected from the group consisting of an encephalomyocarditis virus (EMCV), a hepatitis virus B virus, a hepatitis C virus, a vesicular stomatitis virus (VSV), a pneumovirus, a coronavirus, a coxsackievirus, an influenza virus, a Sendai virus, a cowpox virus and an enterovirus.
9. A method of treating a viral infection in accordance with aspect 8, wherein the influenza virus is an influenza A virus.
10. A method of treating a viral infection in accordance with aspect 6, wherein following the administration of the vector, the subject exhibits an increased rate of viral clearance compared to a control which is not administered the vector.
11. A method of treating a viral infection in accordance with aspect 6, wherein at least one cell of the one or more cells exhibits increased activation of IFN-β compared to a control cell which is does not express the Stat1-CC transgene.
12. A method of treating a viral infection in accordance with aspect 1, wherein the subject exhibits a decreased rate of viral spread among neighboring cells compared to a control that is not administered the vector.
13. A method of treating a viral infection in accordance with aspect 1, wherein the subject exhibits a decreased rate of viral replication compared to a control that is not administered the vector.
14. A method of treating a viral infection in accordance with aspect 1, wherein a cell which expresses the Stat1-CC transgene exhibits enhanced efficiency of activation interferon-responsive genes, compared to a cell of a control which does not express the Stat1-CC transgene
15. A method of treating a viral infection in accordance with aspect 6, wherein the one or more cells which express the Stat1-CC transgene are one or more cells comprised by an organ or tissue selected from the group consisting of pancreas, brain, lung, and heart.
16. A method of treating a viral infection in accordance with aspect 6, wherein the one or more cells which express the Stat1-CC transgene comprise a Stat1-CC transgene product which exhibits prolonged Tyr-701 phosphorylation in response to IFN-γ treatment compared to cells which express wild-type Stat1.
17. A method of treating a viral infection in accordance with aspect 6, wherein the one or more cells which express the Stat1-CC transgene comprise a Stat1-CC transgene product which exhibits prolonged nuclear localization in response to IFN-γ treatment compared to cells which express wild-type Stat1.
18. A method of treating a viral infection in accordance with aspect 6, wherein the one or more cells which express the Stat1-CC transgene exhibit increased IFN efficacy upon administration of IFN, compared to cells which express wild-type Stat1.
19. A method of inducing expression of at least one IFN-responsive gene in at least one cell in vivo, the method comprising administering to a subject a vector comprising a Stat1-CC transgene.
20. A method of inducing increased expression of at least one IFN-responsive gene in vivo in accordance with aspect 19, wherein the vector is an adeno-associated virus (AAV).
21. A method of inducing increased expression of at least one IFN-responsive gene in vivo in accordance with aspect 20, wherein the AAV is an AAV5.
22. A method of inducing increased expression of at least one IFN-responsive gene in vivo in accordance with aspect 19, wherein the vector further comprises a promoter operably linked to the Stat1-CC transgene.
23. A method of inducing increased expression of at least one IFN-responsive gene in vivo in accordance with aspect 22, wherein the promoter operably linked to the Stat1-CC transgene is a CMV-β-actin promoter.
24. A method of inducing increased expression of at least one IFN-responsive gene in vivo in accordance with aspect 22, wherein following the administration of the vector, the subject comprises one or more cells which express the Stat1-CC transgene.
25. A method of inducing increased expression of at least one IFN-responsive gene in vivo in accordance with aspect 24, wherein the at least one cell IFN-responsive gene is at least one type I IFN-responsive gene.
26. A method of inducing increased expression of at least one IFN-responsive gene in vivo in accordance with aspect 25, wherein the at least one type I IFN-responsive gene is selected from the group consisting of an beta2-microglobulin (B2M), guanylate binding protein 1 (GBP1), and interferon regulatory factor 1 (IRF1).
27. A method of inducing increased expression of at least one IFN-responsive gene in vivo in accordance with aspect 24, further comprising administering an IFN to the subject.
28. A method of inducing increased expression of at least one IFN-responsive gene in vivo in accordance with aspect 27, wherein the IFN is an IFN-β.
29. A method of inducing increased expression of at least one IFN-responsive gene in vivo in accordance with aspect 27, wherein the at least one IFN-responsive gene is selected from the group consisting of at least one type I IFN-responsive gene, at least one type II IFN-responsive gene and a combination thereof.
30. A method of inducing increased expression of at least one IFN-responsive gene in vivo in accordance with aspect 29, wherein the type I IFN-responsive gene is selected from the group consisting of B2M, GBP1, and IRF1, and wherein the type II IFN-responsive gene is an ICAM-1
31. A method of treating an interferon-responsive disease, the method comprising administering to a subject in need thereof a vector comprising a Stat1-CC transgene.
32. A method of treating an interferon-responsive disease in accordance with aspect 31, wherein the disease is selected from the group consisting of multiple sclerosis, amyotrophic lateral sclerosis, lupus, hepatitis C infection, a respiratory disorder and a cancer.
33. A method of treating an interferon-responsive disease in accordance with aspect 32, wherein the respiratory disorder is selected from the group consisting of an interstitial lung disease, a malignant mesothelioma, a malignant pleural effusion, and a respiratory infection.
34. A method of treating an interferon-responsive disease in accordance with aspect 32, wherein the cancer is selected from the group consisting of a hairy cell leukemia, a malignant melanoma, a Kaposi's sarcoma, a bladder cancer, a chronic myelocytic leukemia, a kidney cancer, a non-Hodgkin's lymphoma, a lung cancer, an ovarian cancer, and a skin cancer.
35. A method of treating an interferon-responsive disease in accordance with aspect 31, further comprising administering an effective dose of interferon to the subject.
36. A method of treating an interferon-responsive disease in accordance with aspect 35, wherein the effective dose of the interferon is less than an effective dose of the interferon without administering the vector.
37. A method of treating an interferon-responsive disease in accordance with aspect 31, further comprising administering an effective dose of an inducer of interferon expression to the subject.
38. A method of treating an interferon-responsive disease in accordance with aspect 37, wherein the effective dose of the inducer of interferon expression is less than an effective dose of the inducer of interferon expression without administering the vector.
39. A method in accordance with any one of aspects 1-38, wherein the subject is a mammal.
40. A method in accordance with aspect 39, wherein the mammal is a human.
41. A method of protecting a subject from a viral infection, the method comprising administering to a subject a vector comprising a Stat1-CC transgene.
42. A method of protecting a subject from a viral infection in accordance with aspect 41, wherein the vector is an adeno-associated virus (AAV).
43. A method of protecting a subject from a viral infection in accordance with aspect 42, wherein the AAV is an AAV5.
44. A method of protecting a subject from a viral infection in accordance with aspect 41, wherein the vector further comprises a promoter operably linked to the Stat1-CC transgene.
45. A method of protecting a subject from a viral infection in accordance with aspect 44, wherein the promoter operably linked to the Stat1-CC transgene is a CMV-β-actin promoter.
46. A method of protecting a subject from a viral infection in accordance with aspect 44, wherein following the administration of the vector, the subject comprises one or more cells which express the Stat1-CC transgene.

All references cited herein are incorporated by reference, each in its entirety. Applicant reserves the right to challenge any conclusions presented by the authors of any reference.

What is claimed is:

1. A method of inducing increased expression of at least one interferon (IFN)-responsive gene in at least one cell in vivo, the method comprising administering to a subject a vector comprising a Stat1-CC transgene.

2. A method of inducing increased expression of at least one IFN-responsive gene in vivo in accordance with claim 1, wherein following the administration of the vector, the subject comprises one or more cells which express the Stat1-CC transgene.

3. A method of inducing increased expression of at least one IFN-responsive gene in vivo in accordance with claim 2, wherein the one or more cells which express the Stat1-CC transgene are selected from the group consisting of pancreas cells, brain cells, lung cells, and heart cells.

4. A method of inducing increased expression of at least one IFN-responsive gene in vivo in accordance with claim 1, wherein the at least one IFN-responsive gene is selected from the group consisting of at least one type I IFN-responsive gene, at least one type II IFN-responsive gene and a combination thereof.

5. A method of inducing increased expression of at least one IFN-responsive gene in vivo in accordance with claim 4, wherein the at least one type I IFN-responsive gene is selected from the group consisting of an OASbeta2-microglobulin (B2M), guanylate binding protein 1 (GBP1) an Mx-1, and interferon regulatory factor 1 (IRF1), and wherein the type II IFN-responsive gene is an ICAM-1.

6. A method of inducing increased expression of at least one IFN-responsive gene in vivo in accordance with claim 1, further comprising administering an effective dose of an interferon to the subject.

7. A method of inducing increased expression of at least one IFN-responsive gene in vivo in accordance with claim 6, wherein the effective dose of the interferon is less than an effective dose of the interferon without administering the vector.

8. A method of inducing increased expression of at least one IFN-responsive gene in vivo in accordance with claim 6, wherein the IFN is an IFN-β.

9. A method of inducing increased expression of at least one IFN-responsive gene in vivo in accordance with claim 1, wherein the subject is in need of treatment of an infection of a virus which induces a cellular interferon response.

10. A method of inducing increased expression of at least one IFN-responsive gene in vivo in accordance with claim 9, wherein the virus is selected from the group consisting of an encephalomyocarditis virus (EMCV), a hepatitis virus B virus, a hepatitis C virus, a vesicular stomatitis virus (VSV), a pneumovirus, a coronavirus, a coxsackievirus, an influenza virus, a Sendai virus, a cowpox virus and an enterovirus.

11. A method of inducing increased expression of at least one IFN-responsive gene in vivo in accordance with claim 9, wherein the virus is an influenza A virus.

12. A method of inducing increased expression of at least one IFN-responsive gene in vivo in accordance with claim 9, wherein following the administration of the vector, the subject exhibits an increased rate of viral clearance and/or a decreased rate of viral replication compared to a control which is not administered the vector.

13. A method of inducing increased expression of at least one IFN-responsive gene in vivo in accordance with claim 1, wherein the subject is in need of treatment of an interferon-responsive disease.

14. A method of inducing increased expression of at least one IFN-responsive gene in vivo in accordance with claim 13, wherein the interferon-responsive disease is selected from the group consisting of multiple sclerosis, amyotrophic lateral sclerosis, lupus, hepatitis C infection, a respiratory disorder and a cancer.

15. A method of inducing increased expression of at least one IFN-responsive gene in vivo in accordance with claim 14, wherein the respiratory disorder is selected from the group consisting of an interstitial lung disease, a malignant mesothelioma, a malignant pleural effusion, and a respiratory infection.

16. A method of inducing increased expression of at least one IFN-responsive gene in vivo in accordance with claim 14, wherein the cancer is selected from the group consisting of a hairy cell leukemia, a malignant melanoma, a Kaposi's sarcoma, a bladder cancer, a chronic myelocytic leukemia, a kidney cancer, a non-Hodgkin's lymphoma, a lung cancer, an ovarian cancer, and a skin cancer.

17. A method of treating an interferon-responsive disease in a subject, comprising:
inducing increased expression of at least one interferon (IFN)-responsive gene in at least one cell in vivo in accordance with claim 1, and
administering an effective dose of an inducer of interferon expression to the subject.

18. A method of treating an interferon-responsive disease in accordance with claim 17, wherein the effective dose of the inducer of interferon expression is less than an effective dose of the inducer of interferon expression without administering the vector.

19. A method of protecting a subject from a viral infection, the method comprising administering to a subject a vector comprising a Stat1-CC transgene.

20. A method of protecting a subject from a viral infection in accordance with claim 19, wherein the vector is an adeno-associated virus (AAV).


SEQUENCE LISTING (12 sequences)

SEQ ID NO: 1 - DNA, 23 bp, Artificial Sequence
  primer
aagtggagctctctgatccttca

SEQ ID NO: 2 - DNA, 18 bp, Artificial Sequence
  primer
ggcctaccccagcaatga

SEQ ID NO: 3 - DNA, 17 bp, Artificial Sequence
  primer
tgctgcccacccagtga

SEQ ID NO: 4 - DNA, 18 bp, Artificial Sequence
  primer
tgagtgtggtgcctttgc

SEQ ID NO: 5 - DNA, 19 bp, Artificial Sequence
  primer
cgagtggacctgaggaccc

SEQ ID NO: 6 - DNA, 19 bp, Artificial Sequence
  primer
agtgtgagagccgcccttg

SEQ ID NO: 7 - DNA, 20 bp, Artificial Sequence
  primer 8
ctacaggtgtcacccatgcc

SEQ ID NO: 8 - DNA, 21 bp, Artificial Sequence
  primer
gctatcttcccttcctcatcc

SEQ ID NO: 9 - DNA, 21 bp, Artificial Sequence
  primer
cctaagatgacctgcagacgg

SEQ ID NO: 10 - DNA, 21 bp, Artificial Sequence
  primer
tttgacagacttcaccacccc

SEQ ID NO: 11 - DNA, 16 bp, Artificial Sequence
  primer
ctgccttcggtgtcgc

SEQ ID NO: 12 - DNA, 21 bp, Artificial Sequence
  primer
tgggtcgaatcaaagttggag